(19) United States
(12) Patent Application Publication
(10) Pub. No.: US 2022/0000872 A1
(43) Pub. Date: Jan. 6, 2022

(54) METHOD OF ENHANCING IMMUNE-BASED THERAPY

(71) Applicant: OncoTracker, Inc., West Hollywood, CA (US)

(72) Inventors: James Richard BERENSON, Beverly Hills, CA (US); Haiming CHEN, West Hollywood, CA (US)

(21) Appl. No.: 17/289,963

(22) PCT Filed: Oct. 31, 2019
(86) PCT No.: PCT/US2019/059199

§ 371 Date: Apr. 29, 2021

Related U.S. Application Data
(60) Provisional application No. 62/753,666, filed on Oct. 31, 2018.

(51) Int. Cl. A61K 31/519; A61K 35/17; A61K 35/28; A61K 39/395; A61P 35/00

(52) U.S. Cl. CPC A61K 31/519; A61K 35/17; A61P 35/00; A61K 39/3955; A61K 35/28

(57) ABSTRACT

The present invention provides methods of treating and/or inhibiting cancer by administering a JAK1/2 inhibitor (e.g., ruxolitinib). The JAK1/2 inhibitor decreases expression of (or inhibits increased expression of) the checkpoint proteins PD-1, PD-L1, PD-L2, or B7 H3, and/or enhances T-cell killing of tumor cells, and/or enhances the anti-tumor effects of checkpoint inhibitors. The disclosed methods improve the efficacy of immune-based therapies used in treatment of cancer.

### CROSS-REFERENCE TO RELATED APPLICATIONS

This application claims priority to U.S. Provisional Patent Application No. 62/753,666, filed Oct. 31, 2018, the disclosure of which is incorporated by reference herein in its entirety for all purposes.

### FIELD OF THE INVENTION

The invention relates generally to methods of treating or inhibiting cancer. In particular, the invention relates to combination therapy with a JAK1/2 inhibitor and one or more other agents.

### INTRODUCTION

T cells express the checkpoint protein PD-1, which acts as a receptor for two ligands, PD-L1 and PD-L2. When T cells expressing PD-1 encounter PD-L1 or PD-L2, the cytotoxic activity of the T cell is downregulated. Some cancers overexpress PD-L1 or PD-L2 as a mechanism for evading the anti-tumor effects from the immune system.

Inhibitors of PD-1, PD-L1, and PD-L2 can be used to treat cancer. For example, the PD-1 inhibitors pembrolizumab (Keytruda), nivolumab (Opdivo), and cemiplimab (Libtayo) have been shown to be useful in treating several types of cancer, including melanoma of the skin, non-small cell lung cancer, kidney cancer, bladder cancer, head and neck cancers, and Hodgkin lymphoma. The PD-L1 inhibitors atezolizumab (Tecentriq), avelumab (Bavencio), and durvalumab (Imfinzi) have also been shown to be useful in treating cancers, including bladder cancer, non-small cell lung cancer, and Merkel cell skin cancer (Merkel cell carcinoma). These PD-1 and PD-L1 inhibitors and other PD-1 and PD-L1 inhibitors are being studied in further clinical trials for use against various types of cancer.

Similarly, B7-H3 (CD276) is an important immune checkpoint member of the B7 and CD28 families. Induced on antigen presenting cells, B7-H3 plays an important role in the inhibition of T cell function. Importantly, B7-H3 is highly overexpressed on a wide range of human solid cancers and often correlates with both negative prognosis and poor clinical outcome in patients. (See Picarda et al. Molecular Pathways: Targeting B7-H3 (CD276) for Human Cancer Immunotherapy. Clin Cancer Res. 2016 Jul. 15; 22(14): 3425-3431.) A monoclonal antibody against B7-H3 (enoblituzumab) is currently undergoing evaluation as part of clinical trials for treating cancer patients.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. Syn et al. De-novo and acquired resistance to immune checkpoint targeting. The Lancet Oncology. 18 (12): e731-e741 (2017). It acts as an “off” switch when bound to CD80 or CD86 on the surface of antigen-presenting cells resulting in a reduction in anti-tumor effects from immune cells.

Myeloid-derived suppressor cells (MDSCs) play an important role in the regulation of tumor growth and has stimulated the search for a way to therapeutically target these cells, as reviewed in Gabrilovich et al. Myeloid-derived suppressor cells. Cancer Immunol Res. 5:3-8 (2017). Natural killer (NK) cells have been shown to have anti-tumor effects and gene modified versions, such as with a chimeric receptor, have been studied in the treatment of a variety of cancer types.

Immunoreceptor tyrosine-based activation motif (ITAM) molecules have immunostimulatory effects through their effects on Syk family kinases whereas immunoreceptor tyrosine-based inhibition motif (ITIM) molecules are predominantly immune inhibitory through their recruiting effects on tyrosine phosphatases. Compounds to manipulate these molecules have now entered clinical trials for the treatment of cancer patients. Currently, a T-cell immunoglobulin and ITIM inhibitor is being used to treat metastatic cancer as part of a clinical trial. Antibodies, bispecific antibodies, or multispecific antibodies that cross-link ITAM receptors or ITIM receptors have been recently developed.

Chimeric Antigen Receptor T cell (CAR T) therapy is another treatment modality currently being studied for the treatment of a variety of cancers. This cellular-based therapy has shown promising results in a variety of hematologic cancers, including multiple myeloma, lymphoma and acute lymphoblastic leukemia. T-cells are derived from the patient and manipulated in the laboratory so that they contain both a marker for T cell activation and a way to target proteins present specifically on the malignant cells. This dual effect results in anti-tumor effects following infusion of the manipulated T-cells to the patient.

Other cellular therapies that have shown anti-tumor effects include donor lymphocyte infusions as well as allogeneic and autologous hematopoietic cells. Graft-versus-tumor effects have been observed with these cellular products for a variety of different cancer types.

Current immune-based therapies are susceptible to resistance mechanisms related to upregulation of immune checkpoint receptors and their ligands. There is a lack of therapeutic methodologies for overcoming such resistance mechanisms and for enhancing checkpoint-mediated therapies. The present disclosure provides methods for enhancing immune-based therapies and methods for treating and/or inhibiting cancer that address these and other important unmet needs.

### SUMMARY OF THE DISCLOSURE

The invention relates generally to methods of treating or inhibiting cancer. In particular, the invention relates to combination therapy with a JAK1/2 inhibitor and one or more other agents, such as an immune-based therapy.

In one aspect, the disclosure provides a method of inhibiting cancer cell growth, comprising contacting the cancer cell with a JAK1/2 inhibitor or derivative thereof and an immune-based therapy.

In another aspect, the disclosure provides a method of decreasing expression of a checkpoint receptor or ligand by a cell, comprising contacting the cell with a JAK1/2 inhibitor or derivative thereof and an immune-based therapy.

In another aspect, the disclosure provides a method of treating and/or inhibiting cancer in a subject being treated for a cancer with an immune-based therapy, comprising administering the subject an immune-based therapy and a JAK1/2 inhibitor or derivative thereof.

In another aspect, the disclosure provides a method of increasing the efficacy of an immune-based therapy in a subject being treated for a cancer, comprising administering the subject a JAK1/2 inhibitor or derivative thereof in addition to the immune-based therapy being provided to the subject.

In some embodiments, the immune-based therapy is a cell-based therapy.

In some embodiments, the cell-based therapy is selected from the group consisting of a group consisting of T-cell therapy, CAR T-cell therapy, donor lymphocyte infusion, allogeneic hematopoietic cell therapy, autologous hematopoietic cell therapy, and natural killer (NK) cell therapy.

In some embodiments, the immune-based therapy is selected from the group consisting of a bispecific T-cell engager (BiTE) therapy, a monoclonal antibody-based therapy, an antibody-drug conjugate, a PD-1 inhibitor, a PDL-1 inhibitor, a PD-L2 inhibitor, a B7-H3 inhibitor, a CTLA-4 inhibitor, an immunoreceptor tyrosine-based inhibition motif (ITIM) inhibitor, and an immunoreceptor tyrosine-based activation motif (ITAM) stimulatory agent.

In some embodiments, the immune-based therapy is selected from the group consisting of pembrolizumab, nivolumab, cemiplimab, atezolizumab, avelumab, and durvalumab.

In some embodiments, the cancer is a hematological malignancy.

In some embodiments, the hematological malignancy is a B-cell condition or disorder selected from the group consisting of: multiple myeloma (MM), Waldenstrom's macroglobulinemia (WM), chronic lymphocytic leukemia (CLL), B cell non-Hodgkin's lymphoma, plasmacytoma, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B cell lymphoma, splenic lymphoma, mantle cell lymphoma, large cell lymphoma, diffuse mixed cell lymphoma, immunoblastic lymphoma, primary mediastinal B cell lymphoma, pulmonary B cell angiocentric lymphoma, small lymphocytic lymphoma, B cell proliferations of uncertain malignant potential, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

In some embodiments, the cancer is multiple myeloma.

In some embodiments, the multiple myeloma is relapsed or refractory multiple myeloma.

In some embodiments, the cancer is characterized by upregulation of PD-1, PD-L1, PD-L2, and/or B7-H3.

In some embodiments, the JAK1/2 inhibitor is selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, and CHZ868.

In some embodiments, the JAK1/2 inhibitor is ruxolitinib.

In some embodiments, the JAK1/2 inhibitor is intravenously administered to the subject.

In some embodiments, the JAK1/2 inhibitor is orally administered to the subject.

In some embodiments, the subject is being treated with, or has been previously treated with radiation therapy, chemotherapy, transplantation, immunotherapy, hormone therapy, or photodynamic therapy.

In another aspect, the disclosure provides a pharmaceutical composition comprising a JAK1/2 inhibitor and an immune-based therapy.

In another aspect, the disclosure provides a kit comprising a JAK1/2 inhibitor, an immune-based therapy, and instructions for use thereof.

In another aspect, the disclosure provides a JAK1/2 inhibitor for use in the treatment of a cancer characterized by upregulation of one or more of the checkpoint proteins PD-1, PD-L1, PD-L2, and B7-H3.

In some embodiments, the immune-based therapy is not nivolumab or pembrolizumab.

These and other embodiments of the invention are provided by the Detailed Description that follows.

### BRIEF DESCRIPTION OF DRAWINGS

FIG. 1A depicts analysis of PD-L1 gene expression in MM patients with different clinical status. FIG. 1B depicts analysis of PD-L2 gene expression in MM patients with different clinical status.

FIG. 2A depicts analysis of PD-L1 gene expression in CD 138+ and CD138− mononuclear cells of MM patients with progressive disease (PD). FIG. 2B depicts analysis of PD-L1 gene expression in CD 138+ and CD138− mononuclear cells of MM patients with progressive disease (PD).

FIG. 3A demonstrates that ruxolitinib (RUX) down-regulates PD-L1 gene expression of bone marrow mononuclear cells in MM patient. FIG. 3B demonstrates that ruxolitinib (RUX) down-regulates PD-L2 gene expression in bone marrow mononuclear cells in MM patient.

FIG. 4A demonstrates that RUX inhibits PD-L1 gene expression in bone marrow mononuclear cells co-cultured with THP-1 monocytes. FIG. 4B demonstrates that RUX inhibits PD-L2 gene expression in bone marrow mononuclear cells co-cultured with THP-1 monocytes. MM patient #3041 bone marrow mononuclear cells were co-cultured with THP-1 monocytes with or without RUX (from OW to 10 μM) for 48 hours.

FIG. 5A depicts relative PD-L1 gene expression in bone marrow mononuclear cells from MM patient #2188 cultured with (1 μM) or without RUX for 48 hours. FIG. 5B depicts relative PD-L1 gene expression in bone marrow mononuclear cells from MM patient #2188 co-cultured with stromal cells (ATCC, HS-5) on Transwell inserts with (1 μM) or without RUX for 48 hours.

FIG. 5C depicts relative PD-L1 gene expression in bone marrow mononuclear cells from MM patient #2188 cultured with (1 μM) or without RUX for 48 hours. FIG. 5D depicts relative PD-L1 gene expression in bone marrow mononuclear cells from patent number 2188 co-cultured with THP-1 cells on Transwell inserts with (1 μM) or without RUX for 48 hours.

FIG. 6A demonstrates that RUX reduced PD-L1 expression in bone marrow mononuclear cells in MM patients. FIG. 6B. demonstrates that RUX increased dead mononuclear cells in MM patients.

FIG. 7A depicts B7-H3 gene expression in MM patients. The B7-H3 gene expression in bone marrow mononuclear cells from MM patients was determined using quantitative reverse-transcriptase polymerase chain reaction (qRT-PCR). FIG. 7B demonstrates that RUX down-regulates B7-H3 gene expression in primary MM tumor cells. FIG. 7C demonstrates that RUX decreases B7-H3 gene expression in primary MM tumor cells co-cultured with stromal cells. FIG. 7D demonstrates that RUX reduces B7-H3 gene expression in MM cell line co-cultured with THP-1 monocytes.

FIG. 8A demonstrations that the combination of ruxolitinib (RUX) and anti-PD-L1 antibody increases T-cell induction of apoptosis in myeloma tumor cells in vitro. FIG. 8B demonstrates that the combination of ruxolitinib (RUX) and anti-PD-1 antibody increases T-cell induction of apoptosis in myeloma tumor cells in vitro.

FIG. 9A depicts an apoptosis assay of myeloma tumor cells treated with either ruxolitinib (RUX) or anti-PD-L1 antibody alone in vitro. FIG. 9B depicts an apoptosis assay of myeloma tumor cells treated with either ruxolitinib (RUX) or anti-PD-L1 antibody alone in vitro.

FIG. 10A depicts an apoptosis assay of fresh CD138-selected myeloma tumor cells combined with IL-2-stimulated T-cells and treated with ruxolitinib (RUX) in vitro. FIG. 10B depicts Trypan blue staining assay to determine cell death in fresh CD138-selected myeloma tumor cells combined with IL-2-stimulated T-cells and treated with ruxolitinib (RUX) in vitro.

FIG. 11 demonstrates that RUX increased IL-2 gene expression in bone marrow mononuclear cells (BMMCs) from 3 MM patients.

### DETAILED DESCRIPTION

The present inventors have made the surprising discovery that JAK1/2 inhibitors down-regulate key immune checkpoint proteins, including PD-L1, PD-L2 and B7-H3. The present inventors furthermore have discovered that JAK1/2 inhibitors (particularly ruxolitinib) enhance T-cell mediated killing of multiple-myeloma cells, and that JAK1/2 inhibitors (particularly ruxolitinib) enhances the effects of both anti-PD-1 and anti-PD-L1 antibodies on multiple myeloma cells.

The present inventors have previously demonstrated that the JAK1/2 inhibitor ruxolitinib (JAKAFI) is effective in treating hematological malignancies (in particular, multiple myeloma) when combined with a thalidomide derivative such as lenalidomide (REVLIMID) and optionally a steroid or glucocorticoid, as described in U.S. Patent Application Publication No. US 2017/0106003 A1.

Over 140 clinical studies of ruxolitinib have been recorded by the U.S. National Library of Medicine at ClinicalTrials.gov. Yet, ruxolitinib is approved in the United States only for intermediate or high-risk myelofibrosis, including primary myelofibrosis, post-polycythemia vera myelofibrosis, and post-essential thrombocythemia myelofibrosis. Ruxolitinib is not an approved therapy for cancer. Notably, monotherapy with ruxolitinib for multiple myeloma was reported as ineffective in a Phase 1 clinical trial (ClinicalTrials.gov Identifier: NCT00639002).

The disclosure provides methods of enhancing immune-based therapies using JAK1/2 inhibitors. The methods of the disclosure include the use of a JAK1/2 inhibitor (e.g. ruxolitinib) in combination with immune-based therapies. Administration of a JAK1/2 inhibitor enhances an immune-based therapy including, but not limited to, small-molecule and antibody-based therapy, cellular therapy, and gene therapy. The disclosure provides methods for administration of a JAK1/2 inhibitor (e.g. ruxolitinib) in conjunction with donor lymphocyte infusion, allotransplantation, and/or anti-tumor agents. In some embodiments, JAK1/2 inhibitor is administered with immune cells. In some embodiments, the immune cells are T cells, natural killer (NK) cells, or antigen presenting cells (APCs). The immune cells are in some embodiments genetically modified in one or more ways. In some embodiments, the genetic modification of the immune cell provides a targeting receptor (e.g. a chimeric antigen receptor (CAR) or heterologous T-cell receptor (TCR). In some embodiments, the genetic modification of the immune cell enhances the activity of the immune cell. In some embodiments, the genetic modification of the immune cell enhances survival of the immune cell. In some embodiments, the immune cell is a T cell that comprises a CAR, is TCR deficient, or is CD52 deficient. In some embodiments, the disclosure provides methods of treating and/or inhibiting cancer (such as hematological malignancies) using a JAK1/2 inhibitor in combination with one or more of: a CAR-T cell-based therapy, a bispecific T-cell engager (BiTE), a monoclonal antibody-based therapy, an antibody-drug conjugate, a PD-1 or PDL-1 inhibitor, B7-H3 inhibitor, and a CTLA-4 inhibitor. The methods of the disclosure are not limited to cancer. The disclosure provides methods of treating other diseases or conditions. In some embodiments, the disease or condition is an immune-related condition. In some embodiments, the disease or condition related to malfunction of one or more checkpoint inhibitors, such as, without limitation, the PD-1 pathway, B7-H3 signaling, or CTLA-4 signaling.

Without wishing to be bound by theory, it is contemplated that JAK1/2 inhibitors reduce expression of, decrease expression of, or inhibit an increase in expression of certain molecules associated with relapsed or refractory disease and/or resistance to therapeutic agents. In particular, JAK1/2 inhibitors decrease or inhibit an increase in gene expression of PD-L1, PD-L2, and B7-H3 in tumor-derived (e.g. multiple-myeloma derived) cells and associated tissues. Without wishing to be bound by theory, it is contemplated that JAK1/2 inhibitors increase the activity of immune-based therapies. Without wishing to be bound by theory, it is contemplated that JAK1/2 inhibitors affect the tumor microenvironment. Without wishing to be bound by theory, it is contemplated that JAK1/2 inhibitors synergistically enhance tumor killing by T cells (e.g. CAR-T cells). Without wishing to be bound by theory, it is contemplated that JAK1/2 inhibitors synergistically enhance cellular therapy, including without limitation donor lymphocyte infusion, allotransplant, and/or adoptive T-cell therapy.

All publications, patents and patent applications cited herein are hereby incorporated by reference in their entirety.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2, or 1% to a reference quantity, level, value, number, frequency, percentage, dimension, size, amount, weight or length. In particular embodiments, the terms “about” or “approximately” when preceding a numerical value indicate the value plus or minus a range of 15%, 10%, 5%, or 1%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “antibody” refers to an intact antigen-binding immunoglobulin of any kind, or a fragment thereof that itself specifically binds to the antibody's target antigen, and includes, for example, chimeric, humanized, fully human, and bispecific antibodies. The term “BiTE” refers to a bispecific antibody where one arm of the bispecific antibody is an anti-CD3 antigen binding domain.

The terms “immune-based therapy,” “immune-related therapy,” “immunotherapeutic agent,” “immunotherapeutic,” and the like are used herein to generally mean an agent such as a small molecule, antibody, antibody-based molecules (e.g., bispecific or multispecific antibody), biologic drug, virus, cell (e.g., immune cell), or other composition of matter capable of being used therapeutically whose effect on the subject is mediated at least in part by immune-related mechanisms.

The terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any treatment of a disease in a mammal and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest.

As used herein, the phrase “ameliorating at least one symptom of” refers to decreasing one or more symptoms of the disease or condition for which the subject is being treated. In particular embodiments, the disease or condition being treated is a B-cell condition or disorder, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function). In particular embodiments, the disease or condition being treated is a multiple myeloma, wherein the one or more symptoms ameliorated include bone pain.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also include reducing the intensity, effect, symptoms and/or burden of a disease or condition prior to onset or recurrence of the disease or condition.

As used herein, the term “amount” refers to “an amount effective” or “an effective amount” of cells sufficient to achieve a beneficial or desired prophylactic or therapeutic result, including clinical results. In one embodiment, an effect amount refers to the amount of a JAK1/2 inhibitor or a derivative thereof sufficient to prevent, ameliorate one symptom of, or treat a disease, e.g., a B-cell condition or disorder contemplated herein.

A “prophylactically effective amount” refers to an amount of a JAK1/2 inhibitor or a derivative thereof effective to achieve the desired prophylactic result. Typically, but not necessarily, since a prophylactic dose is used in subjects prior to or at an earlier stage of disease, the prophylactically effective amount is less than the therapeutically effective amount.

A “therapeutically effective amount” of a JAK1/2 inhibitor or an immunotherapeutic agent may vary according to factors such as the disease state, age, sex, and weight of the individual, and the agent to elicit a desired response in the individual. A therapeutically effective amount is also one in which any toxic or detrimental effects of the agent are outweighed by the therapeutically beneficial effects. The term “therapeutically effective amount” includes an amount that is effective to “treat” a subject (e.g., a patient).

As used herein, the terms “conditions sufficient,” or “under conditions sufficient,” refer to the conditions for treating the subject, with one or more agents or compositions contemplated herein. In one embodiment, “conditions sufficient” include administering a sufficient amount, e.g., an effective amount of a JAK1/2 inhibitor or an immunotherapeutic agent to a subject in need thereof.

As used herein, the terms “promoting,” “enhancing,” “stimulating,” or “increasing” generally refer to the ability of compositions contemplated herein to produce or cause a greater physiological response (i.e., measurable downstream effect), as compared to the response caused by either vehicle or a control molecule/composition. One such measurable physiological response includes, without limitation, increased cell killing and/or tumor reduction, increased survival, increased treatment efficacy compared to normal, untreated, or control-treated subjects. The physiological response may be increased by at least 5%, 10%, 20%, 30%, 40%, 50%, 60%, 70%, 80%, 90%, 100%, 125%, 150%, 175%, 200%, or greater compared to the response measured in normal, untreated, or control-treated subjects. An “increased” or “enhanced” response or property is typically “statistically significant”, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) that produced by normal, untreated, or control-treated subjects.

As used herein, the terms “decrease” or “lower,” or “lessen,” or “reduce,” or “abate” refers generally to the ability of compositions contemplated to produce or cause a lesser physiological response (i.e., downstream effects), as compared to the response caused by either vehicle or a control molecule/composition. The physiological response, e.g., tumor cell killing, may be decreased by at least 5%, 10%, 20%, 30%, 40%, 50%, 60%, 70%, 80%, 90%, 100%, 125%, 150%, 175%, 200%, or greater compared to the response measured in normal, untreated, or control-treated subjects. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by normal, untreated, or control-treated subject.

“Hematological malignancy” is a type of cancer that affects blood, bone marrow or lymph nodes. Hematological malignancies may derive from either of the two major blood cell lineages: myeloid or lymphoid cell lines. The myeloid cell line normally produces granulocytes, erythrocytes, thrombocytes, macrophages, and mast cells, whereas the lymphoid cell lines produce B-cells, T-cells, natural killer cells, and plasma cells. Lymphomas, lymphocytic leukemias and myeloma are from the lymphoid cell line. Illustrative examples of hematological malignancies that can be treated with compositions contemplated herein include myelomas, leukemias and lymphomas. Other illustrative examples of hematological malignancies that are suitable for treatment in particular embodiments of the methods contemplated herein include, but are not limited to, MM, WM, acute myeloid leukemia (AML), chronic myeloid leukemia (CML), acute lymphatic leukemia (ALL), CLL, Hodgkin's disease, non-Hodgkin lymphoma, myelodysplastic syndrome (MDS) or myeloproliferative diseases. Usually, hematological malignancies do not form solid tumors.

A “subject,” “subject in need of treatment,” “subject in need thereof,” “individual,” or “patient” as used herein, includes any animal that exhibits a symptom of a disease, disorder, or condition that can be treated with compositions contemplated herein. In particular embodiments, the disease, disorder, or condition relates to a hematological malignancy, e.g., multiple myeloma. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate, and, in preferred embodiments, the subject is a human.

The term “relapse” refers to the diagnosis of return, or signs and symptoms of return, of a cancer after a period of improvement or remission.

“Remission,” also known as “clinical remission,” includes both partial and complete remission. In partial remission, some, but not all, signs and symptoms of cancer have disappeared. In complete remission, all signs and symptoms of cancer have disappeared, although cancer still may be in the body.

“Refractory” refers to a cancer that is resistant to, or non-responsive to, therapy with a particular therapeutic agent. A cancer can be refractory from the onset of treatment (i.e., non-responsive to initial exposure to the therapeutic agent), or as a result of developing resistance to the therapeutic agent, either over the course of a first treatment period or during a subsequent treatment period.

The term “agent” refers to a natural or synthetic polypeptide, polynucleotide, carbohydrate, fatty acid, chemical compound, or small organic molecule.

“JAK1/2 inhibitor” refers to an agent that inhibits the activity of a JAK family kinase including JAK1, JAK2, or both JAK1 and JAK2. Some JAK1/2 inhibitors exclusively inhibit JAK1. Some JAK1/2 inhibitors exclusively inhibit JAK2. Some JAK1/2 inhibitors exclusively inhibit both JAK1 and JAK2.

The disclosure contemplates the use of various JAK1/2 inhibitors and derivatives thereof. In some embodiment, the JAK1/2 inhibitor is ruxolitinib (tradenames Jakafi/Jakavi), tofacitinib (tradenames Xeljanz/Jakvinus, formerly known as tasocitinib and CP-690550), oclacitinib (tradename Apoquel), baricitinib (tradename Olumiant), filgotinib (G-146034, GLPG-0634), gandotinib (LY-2784544), lestaurtinib (CEP-701), momelotinib (GS-0387, CYT-387), pacritinib (SB1518), PF-04965842, upadacitinib (ABT-494), peficitinib (ASP015K, JNJ-54781532), fedratinib (SAR302503), cucurbitacin I (JSI-124), and CHZ868, or a derivative thereof.

In some embodiments, the JAK1/2 inhibitor is ruxolitinib (tradenames Jakafi/Jakavi), tofacitinib (tradenames Xeljanz/Jakvinus, formerly known as tasocitinib and CP-690550), oclacitinib (tradename Apoquel), baricitinib (tradename Olumiant), filgotinib (G-146034, GLPG-0634), gandotinib (LY-2784544), lestaurtinib (CEP-701), momelotinib (GS-0387, CYT-387), pacritinib (SB1518), PF-04965842, upadacitinib (ABT-494), peficitinib (ASP015K, JNJ-54781532), fedratinib (SAR302503), cucurbitacin I (JSI-124), and CHZ868.

Ruxolitinib is a drug used in the art for the treatment of intermediate or high-risk myelofibrosis, a type of myeloproliferative disorder that affects the bone marrow, and for polycythemia vera (PCV) when there has been an inadequate response to or intolerance of hydroxyurea. It is also used for the treatment of acute graft versus host disease. The structure of ruxolitinib is as follows:

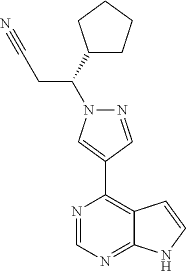 [SMILES: N#CC[C@H](C1CCCC1)n1cc(-c2ncnc3[nH]ccc23)cn1]

The structure, preparation, and characterization of ruxolitinib, and pharmaceutically acceptable salts thereof, are described in, e.g., U.S. Pat. No. 7,598,257 and US Pat. Pub. No. 2008/0312259. A sustained release formulation of ruxolitinib is described in US Pat. Pub. No. 2014/0135350.

In some embodiments, the disclosure provides a combination therapy with a JAK1/2 inhibitor (e.g. ruxolitinib) and an immune-based therapy. In some embodiments, the immune-based therapy comprises an immune cell, a T cell, an NK cell, a chimeric antigen receptor (CAR) T cell (CAR-T), a CAR NK, an antigen presenting cell (APC), a donor lymphocyte, an allotransplant, a bispecific T-cell effector (BiTE), bispecific antibody, or multispecific antibody, a monoclonal antibody, an antibody-drug conjugate (in particular an antibody-drug conjugate with immunomodulatory effect), a PD-1 inhibitor, PD-L1 inhibitor, CTLA-4 inhibitor, and/or B7-H3 inhibitor.

In some embodiments, the methods of the disclosure comprise administering ruxolitinib, thalidomide or a derivative thereof, and a PD-1 inhibitor (e.g. anti-PD-1 antibody) to a subject. In some embodiments, the methods further comprise administering a steroid or a glucocorticoid. The thalidomide or a derivative thereof may be lenalidomide or pomalidomide. Steroid or glucocorticoid useful in the presently disclosed methods include dexamethasone, prednisone, methylprednisolone. In place a PD-1 inhibitor, an anti-PD-L1, anti-PD-L2, anti-B7-H3, or anti-CTLA4 antibody can be used. The methods include treating various cancers including without limitation multiple myeloma. The multiple myeloma may be relapsed or refractory multiple myeloma. In some embodiments, methods comprising administering lenalidomide; dexamethasone, prednisone, methylprednisolone; ruxolitinib; and ipilimumab, pembrolizumab, nivolumab, atezolizumab, durvalumab, or avelumab to a subject suffering from relapsed or refractory multiple myeloma. The treatment advantageously overcomes resistance of relapsed or refractory multiple myeloma to one or more prior treatment selected from the following: lenalidomide; dexamethasone; lenalidomide plus dexamethasone; ruxolitinib plus lenalidomide; or ruxolitinib plus lenalidomide and dexamethasone. The treatment advantageously overcomes resistance of relapsed or refractory multiple myeloma to one or more proteasome inhibitor or chemotherapeutic agent.

In various embodiments, a subject is administered a JAK1/2 inhibitor or derivative thereof to prevent, treat, or ameliorate at least one symptom of a disease or disorder, e.g. a cancer or a B-cell condition or disorder and/or to decrease or prevent expression of one or more of PD-L1, PD-L2 and B7-H3.

In some embodiments, the CAR-T therapy comprises a binding domain which is specific for B-cells, preferably specific for a CD-marker that can be found on B-cell lymphoma such as CD19, CD22, CD20 or CD79a, CD19 being preferred. T-cells that have been genetically engineered to express a CAR (e.g., a T-cell CAR) are exemplified in WO2007/131092.

In some embodiments, the immune-based therapy comprises a monoclonal antibody selected from the group consisting of ipilimumab (anti-CTLA-4), pembrolizumab (anti-PD-1), nivolumab (anti-PD-1), atezolizumab (anti-PD-L1), durvalumab (anti-PD-L1), and avelumab (anti-PD-L1).

In some embodiments, the disclosure provides a method for treating and/or inhibiting a disease or disorder. In some embodiments, the disease or disorder is a cancer. The methods of the present disclosure may include treating any cancer, including, without limitation, acute granulocytic leukemia, acute lymphocytic leukemia, acute myelogenous leukemia, adenocarcinoma, adenosarcoma, adrenal cancer, adrenocortical carcinoma, anal cancer, anaplastic astrocytoma, angiosarcoma, appendix cancer, astrocytoma, basal cell carcinoma, b-cell lymphoma, bile duct cancer, bladder cancer, bone cancer, bone marrow cancer, bowel cancer, brain cancer, brain stem glioma, brain tumor, breast cancer, carcinoid tumors, cervical cancer, cholangiocarcinoma, chondrosarcoma, chronic lymphocytic leukemia, chronic myelogenous leukemia, colon cancer, colorectal cancer, craniopharyngioma, cutaneous lymphoma, cutaneous melanoma, diffuse astrocytoma, ductal carcinoma in situ, endometrial cancer, ependymoma, epithelioid sarcoma, esophageal cancer, Ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors, general, germ cell tumor, gestational trophoblastic disease, glioblastoma multiforme, glioma, hairy cell leukemia, head and neck cancer, hemangioendothelioma, non-Hodgkin lymphoma, Hodgkin lymphoma, Hodgkin's disease, hypopharyngeal cancer, infiltrating ductal carcinoma, infiltrating lobular carcinoma, inflammatory breast cancer, intestinal cancer, intrahepatic bile duct cancer, invasive/infiltrating breast cancer, islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, merkel cell carcinoma, mesenchymal chondrosarcoma, mesenchymous, mesothelioma, metastatic breast cancer, metastatic melanoma, metastatic squamous neck cancer, mixed gliomas, mouth cancer, mucinous carcinoma, mucosal melanoma, multiple myeloma, mycosis fungoides, myelodysplastic syndrome, nasal cavity cancer, nasopharyngeal cancer, neck cancer, neuroblastoma, neuroendocrine tumors, non-Hodgkin lymphoma, non-Hodgkin's lymphoma, non-small cell lung cancer, oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer, ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary tumors, primary central nervous system, prostate cancer, rectal cancer, renal cell carcinoma, renal pelvis cancer, rhabdomyosarcoma, salivary gland cancer, sarcoma, sarcoma, bone, sarcoma, soft tissue, sarcoma, uterine, sinus cancer, skin cancer, small cell lung cancer, small intestine cancer, soft tissue sarcoma, spinal cancer, spinal column cancer, spinal cord cancer, spinal tumor, squamous cell carcinoma, stomach cancer, synovial sarcoma, t-cell lymphoma, testicular cancer, throat cancer, thymoma/thymic carcinoma, thyroid cancer, tongue cancer, tonsil cancer, transitional cell cancer, triple-negative breast cancer, tubal cancer, tubular carcinoma, undiagnosed cancer, ureteral cancer, uterine adenocarcinoma, uterine cancer, uterine sarcoma, vaginal cancer, vulvar cancer.

In some embodiments, the cancer is a solid tumor, such as a melanoma, non-small cell lung cancer, or breast cancer. In some embodiments, the cancer is selected from the group consisting of metastatic melanoma, advanced non-small cell lung cancer (NSCLC), renal cell carcinoma, classical Hodgkin's lymphoma, urothelial cancers, squamous cell cancer of the head and neck, Merkel cell carcinoma, and solid tumors that exhibit microsatellite instability (MSI-H) and mismatch-repair deficiency.

In various embodiments, the disease or disorder is a B-cell condition or disorder. In a particular embodiment, the B-cell condition or disorder is selected from the group consisting of: multiple myeloma (MM), chronic lymphocytic leukemia (CLL), Waldenstrom's macroglobulinemia (WM), and B cell non-Hodgkin's lymphomas (NHL), plasmacytoma, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B cell lymphoma, splenic lymphoma, mantle cell lymphoma, large cell lymphoma, diffuse mixed cell lymphoma, immunoblastic lymphoma, primary mediastinal B cell lymphoma, pulmonary B cell angiocentric lymphoma, small lymphocytic lymphoma, B cell proliferations of uncertain malignant potential, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

In one embodiment, the B-cell condition or disorder is a B cell malignancy. In a particular embodiment, the B-cell condition or disorder is a plasma cell malignancy.

In one embodiment, the B-cell condition or disorder is selected from the group consisting of: MM, WM, CLL, and B-cell non-Hodgkin's lymphoma.

In a certain embodiment, the B-cell condition or disorder is MM.

Compositions (i.e., medicaments) contemplated herein include, but are not limited to pharmaceutical compositions. A “pharmaceutical composition” refers to a formulation of a composition with one or more pharmaceutically acceptable carriers, diluents or excipients generally accepted in the art for the delivery of a compound or drug to a mammal, e.g., humans. In particular embodiments, pharmaceutical compositions comprise a JAK1/2 inhibitor or a derivative thereof, formulated with one or more pharmaceutically-acceptable carriers, diluents, and/or excipients. It will also be understood that, if desired, the compositions of the invention may be administered in combination with other agents as well, such as, e.g., nucleic acids, proteins, small molecules, or pharmaceutically-active agents, adjunct therapies, etc. so long as the desired therapeutic effect is achieved. There is virtually no limit to other reagents that may also be included in the compositions, provided that the additional reagents do not adversely affect the desired cancer therapy.

In particular embodiments, compositions comprise pharmaceutically acceptable formulations with therapeutically effective amounts of JAK1/2 inhibitors or derivatives thereof; or prodrugs, solvates, stereoisomers, racemates, or tautomers of JAK1/2 inhibitors or derivatives thereof, formulated with one or more pharmaceutically acceptable carriers (additives), other active agents, and/or diluents.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compounds contemplated herein exist in free base or acid form and can be converted to their pharmaceutically acceptable salts by treatment with the appropriate inorganic or organic base or acid by methods known to one skilled in the art. “Pharmaceutically acceptable salt” includes both acid and base addition salts. “Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Salts of the compounds of the invention can be converted to their free base or acid form by standard techniques.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In particular embodiments, a pharmaceutical composition contemplated herein is formulated so as to allow the active ingredients contained therein to be bioavailable upon administration of the composition to a subject. In one embodiment, pharmaceutical compositions can be prepared by combining a JAK1/2 inhibitor or derivative thereof, with an appropriate pharmaceutically acceptable carrier, diluent or excipient, and may be formulated into preparations in solid, semi solid, liquid, gels, and microspheres. However, in certain embodiments the subject compounds may be simply dissolved or suspended in sterile water of physiological saline, Ringer's solution, or 0.9% NaCl.

Solid formulations of the compositions contemplated herein, include dragees, capsules, pills and granules, optionally scored or prepared with coatings and shells, such as enteric coatings and other coatings. Solid dosage forms may also be formulated so as to provide slow or controlled release of the compound. Thus, solid formulations could include any material that could provide a desired release profile of the compound, including but not limited to hydroxypropylmethyl cellulose in varying proportions, or other polymer matrices, liposomes and/or microspheres.

Coated, gel, or encapsulating formulations of JAK1/2 inhibitors or derivatives thereof may also be formulated to deliver pulsatile, sustained, or extended release. For example, one method of pulsatile release could be achieved by layering multiple coatings of JAK1/2 inhibitors or derivatives thereof, or by incorporating JAK1/2 inhibitors or derivatives thereof within different regions of the formulation having different release times.

Liquid dosage formulations contemplated herein include pharmaceutically acceptable emulsions, microemulsions, solutions, suspensions, syrups and elixirs. In addition, the liquid dosage formulations may contain inert diluents commonly used in the art, including but not limited to water or other solvents; solubilizing agents and emulsifiers, such as ethyl alcohol, isopropyl alcohol, ethyl carbonate, ethyl acetate, benzyl alcohol, benzyl benzoate, propylene glycol, 1,3-butylene glycol; oils (in particular, cottonseed, groundnut, corn, germ, olive, castor and sesame oils); glycerol; tetrahydrofuryl alcohol; polyethylene glycols; and fatty acid esters of sorbitan, and mixtures thereof.

Suspensions formulations include, without limitation, ethoxylated isostearyl alcohols; polyoxyethylene sorbitol and sorbitan esters; microcrystalline cellulose; aluminum metahydroxide; bentonite; agar-agar; tragacanth; and mixtures thereof.

Injectable depot formulations can be made by forming microencapsulated matrices of the composition in biodegradable polymers. Examples of biodegradable polymers include, but are not limited to polylactide-polyglycolide, poly(orthoesters) and poly(anhydrides). The ratio of composition to polymer and the nature of the particular polymer employed can affect the rate of release of JAK1/2 inhibitors or derivatives thereof from the composition. Depot injectable formulations can also be prepared by entrapping the drug in liposomes or microemulsions.

Proper fluidity of liquid, suspension and other formulations of the compounds can be maintained by the use of coating materials such as lecithin; by the maintenance of the required particle size in the case of dispersions; or by the use of surfactants.

Formulations may also include anti-contamination agents for the prevention of microorganism contamination. Anti-contamination agents may include but are not limited to antibacterial and antifungal agents, for example, paraben, chlorobutanol, phenol sorbic acid, antibiotics, and the like.

Formulations may also be sterilized by, for example, by filtration through a bacteria-retaining filter, or by incorporating sterilizing agents in the form of sterile solid formulations which can be dissolved in sterile water, or some other sterile medium immediately before use or formulation.

Formulations may also be endotoxin free. As used herein, the term “endotoxin free” refers to compositions or formulations that contain at most trace amounts (i.e., amounts having no adverse physiological effects to a subject) of endotoxin, and preferably undetectable amounts of endotoxin. By “substantially free of endotoxin” is meant that there is less endotoxin per dose of cells than is allowed by the FDA for a biologic, which is a total endotoxin of 5 EU/kg body weight per day, which for an average 70 kg person is 350 EU per total dose of cells. In one embodiment, the term “endotoxin free” refers to a composition or formulation that is at least 95%, at least 96%, at least 97%, at least 98%, at least 99%, or 100% endotoxin free. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin in humans can produce fever, a lowering of the blood pressure, and activation of inflammation and coagulation, among other adverse physiological effects. Therefore, it is often desirable to remove most or all traces of endotoxin from drug product containers, because even small amounts may cause adverse effects in humans.

Pharmaceutical compositions may further comprise one or more components that enhance the bioavailability of the active ingredients of the composition, e.g., penetration enhancers, stabilizing agents, and one or more components that provide slow or controlled release of the JAK1/2 inhibitor or derivative thereof in a composition, e.g., biocompatible polymers and/or gels.

In particular embodiments, compositions comprising penetration enhancers will facilitate the delivery of the composition across biological barriers. A “penetration enhancer” or “permeability enhancer” includes a polyol such as polyethylene glycol (PEG), glycerol (glycerin), maltitol, sorbitol etc.; diethylene glycol monoethyl ether, azone, benzalkonium chloride (ADBAC), cetylperidium chloride, cetylmethylammonium bromide, dextran sulfate, lauric acid, menthol, methoxysalicylate, oleic acid, phosphatidylcholine, polyoxyethylene, polysorbate 80, sodium glycholate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

Suitable polyols for inclusion in the solutions include glycerol and sugar alcohols such as sorbitol, mannitol or xylitol, polyethylene glycol and derivatives thereof. In some embodiments the composition further includes a preservative. Accepted preservatives such as benzalkonium chloride and disodium edetate (EDTA) are included in the compositions of the invention in concentrations sufficient for effective antimicrobial action, about 0.0001 to 0.1%, based on the weight of the composition.

In particular embodiments, compositions comprise stabilizers to increase the therapeutic lifetime of the compositions in vivo. Exemplary stabilizers include fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In further embodiments, the chosen stabilizer changes the hydrophobicity of the formulation (e.g., oleic acid, waxes), or improves the mixing of various components in the formulation (e.g., ethanol), affects the moisture level in the formula (e.g., PVP or polyvinyl pyrrolidone), affects the mobility of the phase (substances with melting points higher than room temperature such as long chain fatty acids, alcohols, esters, ethers, amides etc. or mixtures thereof; waxes), and/or improves the compatibility of the formula with encapsulating materials (e.g., oleic acid or wax). In other embodiments, stabilizers are present in sufficient amounts to inhibit the degradation of the JAK1/2 inhibitors or derivatives thereof in a composition. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In particular embodiments, compositions are formulated as controlled release formulations. In general, controlled release drug formulations impart control over the release of drug with respect to site of release and time of release in vivo. Controlled release includes to immediate release, delayed release, sustained release, extended release, variable release, pulsatile release and bi-modal release. Advantages offered by controlled release include: less frequent dosing; more efficient drug utilization; localized drug delivery by placement of a delivery device or formulation at a treatment site in vivo; and the opportunity to administer and release two or more different drugs, each having a unique release profile, or to release the same drug at different rates or for different durations, by means of a single dosage unit.

Controlled release formulations may be made by formulating the compositions with biocompatible polymers, viscosity agents, gels, paints, foams, xerogels, microparticles, hydrogels, nanocapsules, and thermoreversible gels, or combinations thereof. In particular embodiments, the polymer or gels are biodegradable. Release properties are often controlled by the particular combination of polymers or gels used to formulate the composition. These methods are well known in the art.

Exemplary polymers suitable for formulating the inventive compositions include, but are not limited to polyamides, polycarbonates, polyalkylenes (polyethylene glycol (PEG)), polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In particular embodiments, the polymer is an ABA-type or BAB-type triblock copolymers or mixtures thereof, wherein the A-blocks are relatively hydrophobic and comprise biodegradable polyesters or poly(orthoester), and the B-blocks are relatively hydrophilic and comprise polyethylene glycol (PEG). The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof.

Exemplary viscosity agents suitable for use in formulating compositions include, but are not limited to, hydroxypropyl methylcellulose, hydroxyethyl cellulose, polyvinylpyrrolidone, carboxymethyl cellulose, polyvinyl alcohol, sodium chondroitin sulfate, sodium hyaluronate, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, or polyvinylpyrrolidone (PVP: povidone).

Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, glycerin-based gels, glycerin-derived compounds, conjugated, or crosslinked gels, matrices, hydrogels, and polymers, as well as gelatins and their derivatives, and various native and synthetic hydrogel and hydrogel-derived compounds, and any combinations or mixtures thereof.

In a particular embodiment, compositions contemplated herein comprise an effective amount of one or more JAK1/2 inhibitors or derivatives thereof, alone or in combination with one or more other therapeutic agents or modalities. Thus, the compositions may be administered individually or in combination with each other and/or with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, NSAIDs, DMARDs, anti-inflammatories, chemotherapeutics, radiotherapeutics, therapeutic antibodies, or other active and ancillary agents.

In certain embodiments, compositions contemplated herein may be administered in conjunction with any number of chemotherapeutic agents. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, δ-diazo-5-oxo-L-norleucine, doxorubicin and its pegylated formulations, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, δ-mercaptopurine, thiamiprine, thioguanine; pyrimidine analogs such as ancitabine, azacitidine, δ-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; δ-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the compositions contemplated herein. In one embodiment, the compositions contemplated herein are administered with nonsteroidal anti-inflammatory drugs (NSAIDS) including aspirin, ibuprofen, naproxen, methotrexate, sulfasalazine, leflunomide, anti-TNF medications, cyclophosphamide, and mycophenolate.

Other exemplary NSAIDs are chosen from the group consisting of ibuprofen, naproxen, naproxen sodium, COX-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers, cytokine inhibitors, such as the TNF antagonists, adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline. Illustrative examples of therapeutic antibodies suitable for combination with compositions contemplated herein, include but are not limited to, bavituximab, bevacizumab (avastin), bivatuzumab, blinatumomab, conatumumab, daratumumab, duligotumab, dacetuzumab, dalotuzumab, elotuzumab (HuLuc63), gemtuzumab, ibritumomab, indatuximab, inotuzumab, lorvotuzumab, lucatumumab, milatuzumab, moxetumomab, ocaratuzumab, ofatumumab, rituximab, siltuximab, teprotumumab, and ublituximab.

In particular embodiments, the compositions contemplated herein are administered with proteasome inhibitors. The term “proteasome inhibitor” refers to any substance which directly or indirectly inhibits the 20S and/or 26S proteasome or an activity thereof. In particular embodiments, proteasome inhibition is specific, i.e., the proteasome inhibitor inhibits proteasome activity at a concentration that is lower than the concentration of the inhibitor required to produce another, unrelated biological effect. Illustrative examples of proteasome inhibitors that can administered with the compositions described herein include, but are not limited to, bortezomib (Velcade, PS-341), carfilzomib (Kyprolis), oprozomib (ONX 0912), delanzomib (CEP-18770), ixazomib citrate (MLN9708), marizomib (NPI-0052; salinosporamide A), dihydroeponemycin, epoxomicin, ONX-914 (PR-957), syringolin A, TMC-95A, argryin A, disulfiram, epigallocatechin-3-gallate, MG-132, lactacystin, HBX41108, MG-262, MG-115, AM114, MLN2238, AM114, gliotoxin, P005091, PSI, omuralide, AdaAhx3L3VS, 8-hydroxyquinoline hemisulfate salt hemihydrate, and clasto-lactacystin β-lactone.

In certain embodiments, the compositions contemplated herein are administered with steroids, e.g. glucorticoids or glucorticoid receptor agonists. Illustrative examples of glucocorticoids and glucocorticoid receptor agonists suitable for use in the compositions and methods contemplated herein include, but are not limited to, medrysone, alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol, as well as combinations thereof.

In particular embodiments, the compositions contemplated herein are administered with one or more immunomodulatory drugs (IMiDs). Exemplary IMiDs include thalidomide and derivatives thereof. The term “thalidomide” refers to drugs or pharmaceutical formulations comprising the active thalidomide compound 2-(2,6-dioxopiperidin-3-yl)-1H-isoindole-1,3(2H)-dione. Thalidomide derivatives thereof refer to structural variants of thalidomide that have a similar biological activity such as, for example, without limitation, lenalidomide (REVLIIVIID™) ACTIMID™ (Celgene Corporation), and POMALYST™ (Celgene Corporation), and the compounds disclosed in U.S. Pat. No. 5,712,291, WO02068414, and WO2008154252, each of which is incorporated herein by reference in its entirety. Illustrative examples of IMiDs that may be administered with the compositions contemplated herein include, but are not limited to, thalidomide, lenalidomide, pomalidomide, linomide, CC-1088, CDC-501, and CDC-801.

In certain embodiments, the compositions described herein are administered in conjunction with one or more cytokines. A “cytokine” refers to proteins released by one cell population that act on another cell as intercellular mediators. Illustrative examples of such cytokines are lymphokines, monokines, and traditional polypeptide hormones. Included among the cytokines are growth hormones such as human growth hormone, hepatic growth factor; tumor necrosis factor-alpha and -beta; mullerian-inhibiting substance; inhibin; activin; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In particular embodiments, the compositions contemplated herein comprise a concentration of one or more pharmaceutically active ingredients (i.e., a JAK1/2 inhibitor or derivative thereof; and optionally pharmaceutically acceptable salts, prodrugs, solvates, stereoisomers, racemates, or tautomers thereof) of between about 0.01% to about 90%, between about 0.01% to about 50%, between about 0.1% to about 70%, between about 0.1% to about 50%, between about 0.1% to about 40%, between about 0.1% to about 30%, between about 0.1% to about 20%, between about 0.1% to about 10%, or between about 0.1% to about 5%, of each active ingredient, by weight of the composition.

In certain embodiments, the compositions described herein have a concentration of each active pharmaceutical agent between about 1% to about 50%, between about 5% to about 50%, between about 10% to about 40%, or between about 10% to about 30%, of the active ingredient, or pharmaceutically acceptable salt, prodrug, solvate, stereoisomer, racemate, or tautomer thereof, by weight of the composition.

In some embodiments, the formulations have a concentration of active pharmaceutical ingredient of between about 0.1 to about 70 mg/mL, between about 0.5 mg/mL to about 70 mg/mL, between about 0.5 mg/mL to about 50 mg/mL, between about 0.5 mg/mL to about 20 mg/mL, between about 1 mg to about 70 mg/mL, between about 1 mg to about 50 mg/mL, between about 1 mg/mL and about 20 mg/mL, between about 1 mg/mL to about 10 mg/mL, or between about 1 mg/mL to about 5 mg/mL, of the active agent, or pharmaceutically acceptable salt, prodrug, solvate, stereoisomer, racemate, or tautomer thereof, by volume of the formulation.

In one embodiment, the formulations additionally provide an immediate release of one or more pharmaceutically active ingredients (i.e., JAK1/2 inhibitor or derivatives thereof, or pharmaceutically acceptable salts, prodrugs, solvates, stereoisomers, racemates, or tautomers thereof) from the composition, or within 1 minute, or within 5 minutes, or within 10 minutes, or within 15 minutes, or within 30 minutes, or within 60 minutes or within 90 minutes.

In another embodiment, a therapeutically effective amount of at least one pharmaceutically active ingredient is released from the composition immediately, or within 1 minute, or within 5 minutes, or within 10 minutes, or within 15 minutes, or within 30 minutes, or within 60 minutes or within 90 minutes.

In yet another embodiment, a composition is formulated as an extended release formulation. In certain embodiments, diffusion of at least one pharmaceutically active ingredient from the formulation occurs for a time period exceeding 5 minutes, 15 minutes, 30 minutes, 1 hour, 4 hours, 6 hours, 12 hours, 18 hours, 1 day, 2 days, 3 days, 4 days, 5 days, 6 days, 7 days, 10 days, 12 days, 14 days, 18 days, 21 days, 25 days, 30 days, 45 days, 2 months 3 months 4 months 5 months 6 months 9 months or 1 year.

In particular embodiments, a therapeutically effective amount of at least one pharmaceutically active ingredient is released from the formulation for a time period exceeding 5 minutes, 15 minutes, 30 minutes, 1 hour, 4 hours, 6 hours, 12 hours, 18 hours, 1 day, 2 days, 3 days, 4 days, 5 days, 6 days, 7 days, 10 days, 12 days, 14 days, 18 days, 21 days, 25 days, 30 days, 45 days, 2 months, 3 months, 4 months, 5 months, 6 months, 9 months, or 1 year.

In further embodiments, the formulation provides both an immediate release and an extended release formulation. In particular embodiments, the formulation contains a 0.25:1 ratio, a 0.5:1 ratio, a 1:1 ratio, a 1:2 ratio, a 1:3, a 1:4 ratio, a 1:5 ratio, a 1:7 ratio, a 1:10 ratio, a 1:15 ratio, or a 1:20 ratio of immediate release and extended release formulations. In a further embodiment the formulation provides an immediate release of a first pharmaceutically active ingredient and an extended release of a second pharmaceutically active ingredient or another therapeutic agent.

In additional embodiments, the formulation provides a 0.25:1 ratio, a 0.5:1 ratio, a 1:1 ratio, a 1:2 ratio, a 1:3, a 1:4 ratio, a 1:5 ratio, a 1:7 ratio, a 1:10 ratio, a 1:15 ratio, or a 1:20 ratio of immediate release and extended release formulations of one or more pharmaceutically active ingredients.

The combination of immediate release, delayed release and/or extended release compositions or formulations may be combined with other pharmaceutical agents, as well as the excipients, diluents, stabilizers, carrier agents and other components disclosed elsewhere herein. As such, depending upon the components of a composition, the thickness or viscosity desired, or the mode of delivery chosen, alternative aspects of the embodiments disclosed herein are combined with the immediate release, delayed release and/or extended release embodiments accordingly.

Additional methods of formulating compositions are known to the skilled artisan, for example, as described in the Physicians Desk Reference, 62nd edition. Oradell, N.J.: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, Md.: Lippincott Williams & Wilkins, 2000; and The Merck Index, Fourteenth Edition. Whitehouse Station, N.J.: Merck Research Laboratories, 2006; each of which is hereby incorporated by reference in relevant parts.

In particular embodiments, a method of treating a subject with a disease or disorder or is contemplated comprising administering to the subject a JAK1/2 inhibitor or a derivative thereof. In some embodiments, the subject has, or is identified as having, a tumor that has one or more of high PD-L1 level or expression, high PD-L2 level or expression, high B7-H3 level or expression, or high CTLA-4 level or expression. In some embodiments, the methods described herein further include identifying a subject based on having a tumor that has one or more of high PD-L1 level or expression, high PD-L2 level or expression, high B7-H3 level or expression, or high CTLA-4 level or expression.

Compositions contemplated herein may be administered as one or more solids, semi-solids, gels, or liquids, or combination thereof. For example, a JAK1/2 inhibitor or derivative thereof and other pharmaceutically active agents may be individually formulated for intravenous administration in a liquid dosage form or for oral administration as a single tablet or capsule or as a combination of one or more tablets, capsules, or other dosage forms. The specific amount/dosage regimen will vary depending on the weight, gender, age and health of the individual; the formulation, the biochemical nature, bioactivity, bioavailability and the side effects of the agents and the number and identity of the agents in the complete therapeutic regimen.

As used herein, the terms “administering,” “administer,” or “administration” refer to deliver one or more compounds or compositions to a subject parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration.

In particular embodiments, an agent or composition is administered parenterally, optionally by intravenous administration or oral administration to a subject.

In various embodiments, the development of suitable dosing and treatment regimens for using the particular compositions contemplated herein in a variety of treatment regimens including, e.g., oral, parenteral, intravenous, intranasal, and intramuscular administration and formulation, is well known in the art. In certain embodiments, a JAK1/2 inhibitor is administered intravenously to a subject. In particular embodiments, a JAK1/2 inhibitor is administered intramuscularly to a subject. In some embodiments, a JAK1/2 inhibitor is administered sublingually to a subject. In particular embodiments, a JAK1/2 inhibitor is administered subcutaneously to a subject.

In particular embodiments, a JAK1/2 inhibitor or derivative thereof is administered orally to a subject. The agent can be administered to the subject at a dose in the range of about 1-100 mg, about 1-50 mg, about 50-100 mg, about 1-5 mg, about 5-10 mg, about 10-15 mg, about 15-20 mg, about 20-30 mg, about 30-40 mg, about 40-50 mg, about 50-60 mg, about 60-70 mg, about 70-80 mg, about 80-90 mg, or about 90-100 mg or more. In certain embodiments, the agent is administered in a dose of about 1 mg, about 2 mg, about 2.5 mg, about 3 mg, about 4 mg, about 5 mg, about 6 mg, about 7 mg, about 8 mg, about 9 mg, about 10 mg, about 15 mg, about 20 mg, about 25 mg, about 50 mg, or about 100 mg or more. In some embodiments of the invention, an oral dose of an agent is administered to the subject at least once in a treatment cycle, at least once in a 28-day treatment cycle, at least once a week, at least once every other day, at least once a day, or at least twice a day.

In particular embodiments, a JAK1/2 inhibitor or a derivative thereof is administered intravenously. The agent can be administered intravenously at a dose of about 0-100 mg, about 1-50 mg, about 50-100 mg, about 1-10 mg, about 10-20 mg, about 20-30 mg, about 30-40 mg, about 40-50 mg, about 50-60 mg, about 60-70 mg, about 70-80 mg, about 80-90 mg, or about 90-100 mg or more. In certain embodiments, the intravenous dose of agent is about one mg, about two mg, about three mg, about four mg, about five mg, about six mg, about seven mg, about eight mg, about nine mg, about ten mg, about 15 mg, about 20 mg, about 25 mg, about 30 mg, about 35 mg, about 40 mg, about 45 mg, about 50 mg, about 60 mg, about 70 mg, about 80 mg, about 90 mg, or about 100 mg or more. Doses of agents can be delivered intravenously in any pharmaceutically suitable vehicles for injection or infusion known in the art.

In some embodiments, the agent can be administered intravenously at a dose of about 0-100 mg/m², about 1-50 mg/m², about 50-100 mg/m², about 1-10 mg/m², about 10-20 mg/m², about 20-30 mg/m², about 30-40 mg/m², about 40-50 mg/m², about 50-60 mg/m², about 60-70 mg/m², about 70-80 mg/m², about 80-90 mg/m², or about 90-100 mg/m² or more. In certain embodiments, the intravenous dose of agent is about one mg/m², about two mg/m², about three mg/m², about four mg/m², about five mg/m², about six mg/m², about seven mg/m², about eight mg/m², about nine mg/m², about ten mg/m², about 15 mg/m², about 20 mg/m², about 25 mg/m², about 30 mg/m², about 35 mg/m², about 40 mg/m², about 45 mg/m², about 50 mg/m², about 60 mg/m², about 70 mg/m², about 80 mg/m², about 90 mg/m², or about 100 mg/m² or more.

In some embodiments, the agent can be administered intravenously at a dose of about 0-10 mg/kg, about 0-5 mg/kg, about 5-10 mg/kg, about 0-1 mg/kg, about 1-2 mg/kg, about 2-3 mg/kg, about 3-4 mg/kg, about 4-5 mg/kg, about 5-6 mg/kg, about 6-7 mg/kg, about 7-8 mg/kg, about 8-9 mg/kg, or about 9-10 mg/kg or more. In certain embodiments, the intravenous dose of agent is about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about one mg/kg, about two mg/kg, about three mg/kg, about four mg/kg, about five mg/kg, about six mg/kg, about seven mg/kg, about eight mg/kg, about nine mg/kg, or about ten mg/kg or more.

In some embodiments, a JAK1/2 inhibitor or a derivative thereof is administered at least once during a treatment cycle. In some embodiments, a JAK1/2 inhibitor or a derivative thereof is administered to the subject on the same days. In some embodiments, a JAK1/2 inhibitor or a derivative thereof is administered to the subject on the different days. In some embodiments, a JAK1/2 inhibitor or a derivative thereof is administered to the subject on the same days and on different days according to treatment schedules.

In particular embodiments, an agent is administered to the subject over one or more treatment cycles. A treatment cycle can be at least two, at least three, at least four, at least five, at least six, at least seven, at least 14, at least 21, at least 28, at least 48, or at least 96 days or more. In one embodiment, a treatment cycle is 28 days. In certain embodiments, the agents are administered over the same treatment cycle or concurrently over different treatment cycles assigned for each agent. In various embodiments, the treatment cycle is determined by a health care professional based on conditions and needs of the subject.

In some embodiments, an agent is administered on at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle. In particular embodiments, an agent is administered to a subject once a day. In other particular embodiments, an agent is administered twice a day. In certain embodiments an agent is administered more than twice a day.

In particular embodiments, an agent is administered on day 1, day 2, day 8, day 9, day 15, and day 16 of a 28-day treatment cycle. In some embodiments, a JAK1/2 inhibitor or a derivative thereof is administered on day 1, day 2, day 8, day 9, day 15, and day 16 of a 28-day treatment cycle.

The number of times a composition is administered to a subject in need thereof depends on the discretion of a medical professional, the disorder, the severity of the disorder, and the subject's response to the formulation. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; or, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In various embodiments, the present invention contemplates therapies comprising administering one or more JAK1/2 inhibitors to a subject.

The combination therapies disclosed herein can result in one or more of: an increase in antigen presentation, an increase in effector cell function (e.g., one or more of T cell proliferation, IFN-alpha secretion or cytolytic function), inhibition of regulatory T cell function, an effect on the activity of multiple cell types (such as regulatory T cell, effector T cells and NK cells), an increase in tumor infiltrating lymphocytes, an increase in T-cell receptor mediated proliferation, and a decrease in immune evasion by cancerous cells.

In one embodiment, the methods contemplated herein comprise treating or preventing cancer in a subject comprising administering to the subject one or more JAK1/2 inhibitors or a derivative thereof.

In a still further aspect, the invention provides methods of treating or preventing or delaying cancer or a B-cell mediated condition disorder. The method includes administering to a subject in which such treatment or prevention or delay is desired, a composition of the invention in an amount sufficient to treat, prevent, or delay a tumorigenic or immunoregulatory condition in the subject. In some embodiments, the subject is a human. In other embodiments, the subject is a non-human mammal. In some embodiments, administration of the composition of the invention reduces or prevents expression of PD-L1, PD-L2, or B7-H3 in the subject, which may result in one or more of cell death; apoptosis; and inhibition, reduction, or cessation of cell proliferation.

Illustrative examples of immune-related conditions or disorders suitable for treatment with the compositions or methods contemplated herein include, without limitation, autoimmune diseases involving inappropriate B cell activity and B cell lymphomas. B cell lymphomas include, without limitation, MM, plasmacytoma, WM, CLL, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B-cell lymphoma, splenic lymphoma, mantle cell lymphoma, large cell lymphoma, diffuse mixed cell lymphoma, immunoblastic lymphoma, primary mediastinal B-cell lymphoma, pulmonary B-cell angiocentric lymphoma, small lymphocytic lymphoma, lymphomatoid granulomatosis and post-transplant lymphoproliferative disorder, primary or immunocyte-associated amyloidosis, and monoclonal gammopathy of undetermined significance (MGUS).

Illustrative examples of B cell-related conditions or disorders suitable for treatment with the compositions or methods contemplated herein include, without limitation, disorders that are autoimmune in nature such as, for example, systemic lupus erythematosus, rheumatoid arthritis, myasthenia gravis, autoimmune hemolytic anemia, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, heavy-chain disease, and rapidly progressive glomerulonephritis.

Illustrative examples of hematological malignancies suitable for treatment with the compositions and methods contemplated herein include, but are not limited to MM, WM, leukemia, or lymphoma. Leukemias can include, but are not limited to, ALL, AML, CLL, CIVIL, and acute monocytic leukemia. Lymphomas can include, but are not limited to, Hodgkin's lymphomas, such as nodular sclerosis Hodgkin's lymphoma, mixed cellularity subtype Hodgkin's lymphoma, Lymphocyte rich Hodgkin's lymphoma, and lymphocyte depleted Hodgkin's Lymphoma; and non-Hodgkin's lymphoma, such as diffuse large B-cell lymphoma, primary mediastinal B-cell lymphoma, follicular lymphoma, CLL, mantle cell lymphoma, marginal zone B-cell lymphomas, Burkitt lymphoma, lymphoplasmacytic lymphoma, primary central nervous system lymphoma, T-cell lymphomas, and WM. Plasma cell dyscrasias include, but are not limited to, multiple myeloma.

In various embodiments, methods of preventing or decreasing PD-L1, PD-L2, or B7-H3 expression is also contemplated, comprising administering to a subject, one or more JAK1/2 inhibitors or a derivative thereof. Preventing or decreasing PD-L1, PD-L2, or B7-H3 expression to increase the efficacy of or prevent development of resistance to immune-based therapies. Without wishing to be bound by any particular theory, it is contemplated that preventing or decreasing PD-L1, PD-L2, or B7-H3 expression will enhance other therapies that target PD-1, PD-L1, PD-L2, or B7-H3, since if PD-L1, PD-L2, or B7-H3 expression is decrease, immune evasion via checkpoint pathways is decreased. Immune-based therapies include, but are not limited to, CAR-T cell-based therapy, a bispecific T-cell engager (BiTE), a monoclonal antibody-based therapy, an antibody-drug conjugate, a PD-1 or PDL-1 inhibitor, and a CTLA-4 inhibitor.

In some embodiments, a JAK1/2 inhibitor is administered to a subject receiving an immune-based therapy. In some embodiments, a JAK1/2 inhibitor is administered to a subject before, during, or after, administration of an immune-based therapy. In some embodiments, the immune-based therapies include, but are not limited to, small-molecule or antibody-based therapy, cellular therapy, and gene therapy. Exemplary immune-based therapies include donor lymphocyte infusion and allotransplantation. In some embodiments, the immune-based therapy comprises immune cells. In some embodiments, the immune cells are T cells, natural killer (NK) cells, or antigen presenting cells (APCs). The immune cells are in some embodiments genetically modified in one or more ways. In some embodiments, the genetic modification of the immune cell provides a targeting receptor (e.g. a chimeric antigen receptor (CAR) or heterologous T-cell receptor (TCR)). In some embodiments, the genetic modification of the immune cell enhances the activity of the immune cell. In some embodiments, the genetic modification of the immune cell enhances survival of the immune cell. In some embodiments, the immune cell is a T cell that comprises a CAR, is TCR-deficient, or is CD52-deficient.

In some embodiments, a JAK1/2 inhibitor is administered to a subject receiving a cellular therapy. In some embodiments, a JAK1/2 inhibitor is administered to a subject receiving a chimeric receptor (e.g., CAR or TCR) T cell-based therapy. Exemplary method of treatment with chimeric receptor T cell including those disclosed in, e.g., International Patent Publication No. WO2018187332A1, the disclosure of which is incorporated herein in its entirety.

In some embodiments, the chimeric receptor targets a tumor antigen.

In some embodiments, the chimeric receptor targets a tumor antigen selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD 123, CD 133, CD 138, CD 19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, UPV-specific antigen, human telom erase reverse transcriptase, IGFI receptor, IGF-II, IL-11Ralpha, IL-13R-a2, Influenza Virus-specific antigen; CD38, insulin growth factor (IGFl)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-AL major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, KG2D, Nkp30, NY-ESO-1, p53, PAP, prostase, prostate specific antigen (PSA), prostate-carcinoma tumor antigen-1 (PCTA-1), prostate-specific antigen protein, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface markers.

In some embodiments, the chimeric receptor specifically targets CD19. In some embodiments, the chimeric receptor is a chimeric antigen receptor (CAR). In some embodiments, the chimeric receptor is a T cell receptor (TCR).

In some embodiments, the JAK1/2 inhibitor is administered at the same time or within one week after the administration of the immune cell. In some embodiments, the chemotherapeutic agent is administered continuously or intermittently for at least 1, 2, 3, 4, or 5 weeks before administering the immune cell. In some embodiments, the immune cells (e.g., T cells) can be administered at a therapeutically effective amount. In some embodiments, administration of ruxolitinib decreases the therapeutically effective amount of the immune cell by at least about 5%, 10%, 15%, 20%, 25%, 30%, 35%, 40%, 45%, or 50%. In some embodiments, administration of ruxolitinib increases therapeutic effectiveness of the immune cell by at least about 5%, 10%, 15%, 20%, 25%, 30%, 35%, 40%, 45%, or 50%; or by 1.5-fold, 2-fold, or 3-fold.

Immune-based therapy include, but are not limited to, an immune cell, a T cell, an NK cell, a chimeric antigen receptor (CAR) T cell (CAR-T), a CAR NK, an antigen presenting cell (APC), a donor lymphocyte, an allotransplant, a bispecific T-cell effector (BiTE), bispecific antibody, or multispecific antibody, a monoclonal antibody, an antibody-drug conjugate (in particular an antibody-drug conjugate with immunomodulatory effect), a PD-1 inhibitor, PD-L1 inhibitor, CTLA-4 inhibitor, and/or B7-H3 inhibitor.

In some embodiments, the immune-based therapy comprises an inhibitor of PD-1, an inhibitor of PD-L1, or an inhibitor of PD-L2. In some embodiments, the immune-based therapy comprises a monoclonal antibody, bispecific antibody, or chimeric antigen receptor T cell specific for PD-1, PD-L1, or PD-L2. In some embodiments, the immune-based therapy comprises pembrolizumab, nivolumab, or cemiplimab. In some embodiments, the immune-based therapy comprises atezolizumab, avelumab, or durvalumab.

In some embodiments, the immune-based therapy comprises a monoclonal antibody, bispecific antibody, or chimeric antigen receptor T cell specific for B7-H3 (CD276). In some embodiments, the immune-based therapy comprises enoblituzumab.

In some embodiments, the immune-based therapy comprises a monoclonal antibody, bispecific antibody, or chimeric antigen receptor T cell specific for CTLA-4 (cytotoxic T-lymphocyte-associated protein 4), also known as CD152 (cluster of differentiation 152).

In some embodiments, the immune-based therapy comprises an immune cell, e.g., a myeloid-derived suppressor cell (MDSC), natural killer (NK) cell, or T cell. In some embodiments, the immune cell comprises an engineered T cell receptor or chimeric antigen receptor.

In some embodiments, the immune-based therapy comprises an antibody, bispecific antibody, or multispecific antibody that cross-links ITAM receptors or ITIM receptors.

In some embodiments, the immune-based therapy comprises a Chimeric Antigen Receptor T-cell (CAR T-cell). In some embodiments, the immune-based therapy comprises tisagenlecleucel (KYMIZIAH). In some embodiments, the immune-based therapy comprises axicabtagene ciloleucel (YESCARTA).

Exemplary immune-based therapies of the present disclosure include, without limitation, ipilimumab, pembrolizumab, nivolumab, atezolizumab, durvalumab, avelumab, BMS-936559, and enoblituzumab. In an embodiment, the disclosure provides a method of treating a subject suffering from late-stage melanoma, comprising administering ruxolitinib and ipilimumab. In an embodiment, the disclosure provides a method of treating a subject suffering from inoperable or metastatic melanoma, metastatic non-small cell lung cancer (NSCLC), head and neck squamous cell carcinoma (HNSCC), or classical Hodgkin's lymphoma, comprising administering ruxolitinib and pembrolizumab. In an embodiment, the disclosure provides a method of treating a subject suffering from inoperable or metastatic melanoma, comprising administering ruxolitinib, nivolumab, and ipilimumab. In an embodiment, the disclosure provides a method of treating a subject suffering from metastatic squamous non-small cell lung cancer, comprising administering ruxolitinib and nivolumab, optionally with or after platinum-base drugs. In an embodiment, the disclosure provides a method of treating a subject suffering from renal cell carcinoma, comprising administering ruxolitinib and nivolumab. In an embodiment, the disclosure provides a method of treating a subject suffering from locally advanced or metastatic urothelial carcinoma, comprising administering ruxolitinib and atezolizumab. In an embodiment, the disclosure provides a method of treating a subject suffering from NSCLC or advanced metastatic urothelial bladder, comprising administering ruxolitinib and durvalumab. In an embodiment, the disclosure provides a method of treating a subject suffering from metastatic Merkel-cell carcinoma (MCC), comprising administering ruxolitinib and avelumab. In an embodiment, the disclosure provides a method of treating a subject suffering from advanced cancer that expresses B7-H3 in the tumor and/or tumor-associated vasculature, comprising administering ruxolitinib and enoblituzumab. In any of the foregoing, one or more other JAK1/2 inhibitors can be used in place of ruxolitinib.

In some embodiments, a JAK1/2 inhibitor is administered to a subject receiving donor lymphocyte infusion. Donor leukocyte infusion (DLI) has several indications after both myeloablative and non-myeloablative allogeneic stem cell transplantation (SCT). It is predominately used to treat and prevent relapse after SCT by exploiting the graft-versus-tumor effect (GVT) of donor-derived T cells. Administration of a JAK1/2 inhibitor (e.g., ruxolitinib) enhances the graft-versus-tumor effect of donor leukocyte infusion. In some embodiments, the disclosure provides a method of treating and/or preventing relapse in a subject suffering from CIVIL, ALL, NHL, HL, or MM, comprising administering ruxolitinib and DLI. In some embodiments, the methods of the disclosure comprise administering ruxolitinib, DLI, and one or more chemotherapeutic agents.

In some embodiments, a JAK1/2 inhibitor is administered to a subject receiving allotransplant. Allotransplant, also referred to as allogeneic stem cell transplantation, involves transferring stem cells from a healthy person (the donor) to the subject. In some cases, allotransplant is used after high-intensity chemotherapy or radiation. Allotransplant may trigger a graft versus tumor effect. Administration of a JAK1/2 inhibitor (such as ruxolitinib) enhances the graft-versus-tumor effect of allotransplant. In some embodiments, the disclosure provides a method of performing an allotransplant, comprising administering ruxolitinib before, during, or after the allotransplant. In some embodiments, the methods of the disclosure comprise administering ruxolitinib, allotransplant, and one or more chemotherapeutic agents.

In some embodiments, a JAK1/2 inhibitor is administered to a subject undergoing a therapy that targets PD-1, PD-L1, PD-L2, B7-H3, or CTLA4. In certain embodiments, the therapy that targets PD-1, PD-L1, PD-L2, B7-H3, or CTLA4 comprises administering a therapeutic agent that binds to PD-1, PD-L1, PD-L2, B7-H3, or CTLA4. In some embodiments, administration of the JAK1/2 inhibitor decreases the expression of PD-L1, PD-L2, B7-H3, or CTLA4 by tumor cells or tumor-associated immune cells by about 5%, about 10%, about 15%, about 20%, about 25%, about 30%, about 20%, about 25%, about 30%, about 35%, about 40%, about 45%, about 50%, about 55%, about 60%, about 65%, about 70%, about 75%, about 80%, about 85%, about 90%, about 95%, about 100%, about 1.5-fold, about 2-fold, about 2.5-fold, about 3-fold, about 4-fold, about 5-fold, about 10-fold, about 20-fold, about 30-fold, about 40-fold, about 50-fold, about 100-fold, or greater than 100-fold (including all ranges and values in-between) as compared to the expression PD-1, PD-L1, PD-L2, B7-H3, or CTLA4 by tumor cells or tumor-associated immune cells in the absences of the JAK1/2 inhibitor.

In various embodiments, the methods contemplated herein comprise increasing the efficacy of a therapy in a subject being treated for cancer (e.g., a B-cell condition or disorder) comprising: administering the subject a JAK1/2 inhibitor or a derivative thereof in addition to the existing treatment being provided to the subject. Therapies for cancer include, but are not limited to, radiation therapy, chemotherapy, transplantation, immune-based therapy, proteasome inhibitors, immunomodulatory agents, hormone therapy, or photodynamic therapy.

In some embodiments, the disclosure provides methods of enhancing therapies directed against myeloid-derived suppressor cells (MDSCs), comprising administering a JAK1/2 inhibitor. The fact that MDSC play an important role in the regulation of tumor growth has stimulated the search for a way to therapeutically target these cells, as reviewed in Gabrilovich et al. Myeloid-derived suppressor cells. Cancer Immunol Res. 5:3-8 (2017). In some embodiments, the methods comprising administering a JAK1/2 inhibitor and an immune-related therapy selected from a chemotherapeutic (e.g., low doses of gemcitabine and 5-fluorouracil), an agent targeting the TNF-related apoptosis-inducing ligand (TRAIL) receptor, a peptibody consisting of S100A9-derived peptides conjugated to antibody Fc, a PDE-5 inhibitor (e.g., tadalafil), a triterpenoid, a COX-2 inhibitor, a histone deactylase (HDAC) inhibitor (e.g., the Class I HDAC inhibitor entinostat), all-trans-retinoic acid (ATRA), a STAT3 inhibitor, and a phospholipid phosphatidylserine (PS) targeting antibody.

In some embodiments, the disclosure provides methods of enhancing therapies using NK cells, comprising administering a JAK1/2 inhibitor. In some embodiments, the NK cells are gene modified, such as with a chimeric receptor. In some embodiments, the methods comprising administering a JAK1/2 inhibitor and an NK cell, optionally a gene-modified NK cell. In some embodiments, the NK cell is a CAR-modified NK cell.

In some embodiments, the disclosure provides methods of enhancing therapies directed against an immunoreceptor tyrosine activation motif (ITAM) and/or immunoreceptor tyrosine activation motif (ITIM), comprising administering a JAK1/2 inhibitor. For example, administration of a JAK1/2 inhibitor in some embodiments enhances the therapeutic activity of antibodies, bispecific antibodies, or multispecific antibodies that cross-link ITAM receptors or ITIM receptors.

In some embodiments, the disclosure provides methods of enhancing an immune-based antibody therapy, comprising administering a JAK1/2 inhibitor.

In certain embodiments, one or more JAK1/2 inhibitors are administered to a subject to increase the efficacy of a therapy or treatment for a disease or disorder that is autoimmune in nature. Therapies for conditions that are autoimmune in nature include, but are not limited to, corticosteroids (e.g. prednisone, prednisolone and methylprednisolone), disease-modifying antirheumatic drugs (DMARDs; e.g., methotrexate, hydroxycholorquine, sulfasalazine, leflunomide, cyclophosphamide and azathioprine), and biologics (e.g., tocilzumab, cerolizumab, etanercept, adalimumab, anakinra, abatacept, infliximab, rituximab), nonsteroidal anti-inflammatory drugs (NSAIDS; e.g. aspirin, ibuprofen, and naproxen), acetylcholinesterase inhibitors (e.g., physostigmine, neostigmine, pyridostigmine, ambenonium, demecarium, rivastigmine, phenanthrene, galantamine, donepezil, tacrine, and edrophonium), cytostatics (e.g., folic acid analogs such as methotrexate, purine analogs such as azathioprine and mercaptopurine, and pyrimidine analogs such as fluorouracil), drugs that act on immunophilins (e.g. ciclosporin, tacrolimius, and sirolimius), and interferons such as IFN-beta.

In various embodiments, one or more JAK1/2 inhibitors are administered to a subject to increase the efficacy of a therapy or a treatment for a B cell condition or disorder that is a hematological malignancy. Therapies for hematological malignancies include, but are not limited to, radiation therapy and chemotherapy (e.g. combination chemotherapy such as MOPP (combination of Mustargen, Oncovin (also known as vincristine), prednisone and procarbazine (also known as Matulane)), ABVD (combination of adriamycin, bleomycin, vinblastine, and dacarbazine), and CHOP (cyclophosphamide, doxorubicin, vincristine, prednisolone); treatment with alkylating agents such as, melphalan, cyclophosphamide, nitrosoureas, tetrazines, aziridines, cisplatins and derivatives, and non-classical alkylating agents such as bendamustine; cytostatics (e.g., folic acid analogs such as methotrexate, purine analogs such as azathioprine and mercaptopurine, and pyrimidine analogs such as fluorouracil); treatment with anti-microtuble agents such as vincristine and vinblastine, taxanes, pacilitaxel, docetaxel, and ligan; treatment with topoisomerase inhibitors such as irinotecan, topotecan, camptothecin, etoposide, doxorubicin, mitoxantrone, and teniposide, proteasome inhibitors such as bortezomib, carfilzomib and ixazomib, and immunomodulatory agents such as thalidomide, lenalidomide and pomalidomide.

Certain embodiments contemplate that an increase in the efficacy of a therapy can be readily determined and/or identified by one skilled in the art. In some embodiments, an improvement of the efficacy of a therapy for disease or disorder is an improvement, an alleviation, an amelioration, and/or a reduction of at least one sign or symptom of the disease or disorder being treated, as compared to the therapy without the one or more JAK1/2 inhibitors. In some embodiments, a subject who is receiving a therapy for a disease or disorder is administered a JAK1/2 inhibitor, and at least one sign or symptom of the disease or disorder is further reduced by the therapy, as compared to treatment with the therapy alone. Symptoms of a disease or disorder include, but are not limited to, generalized weakness and fatigue, anemia, dizziness, frequent or unexplained fever and infection, weight loss or loss of appetite, excessive and unexplained bruising, breathlessness, enlarged lymph nodes, liver, or spleen, pitting edema, joint inflammation, blood clots, skin rash, jaundice, itchy skin, joint pain, insomnia, heat sensitivity, muscle weakness, tremors, paralysis, difficulty speaking, difficulty breathing.

In particular embodiments, improved efficacy of a therapy includes an alleviation, abatement, amelioration, and/or reduction on at least one unwanted side effect of the therapy. Thus, in certain embodiments, one or more JAK1/2 inhibitors are administered to a subject receiving a therapy for a disease or disorder to abate, alleviate, ameliorate, and/or reduce at least one unwanted side effect of the therapy. Examples of unwanted side effects will be readily identified by those of skill in the art, and include, but are not limited to, increased risk or incidence of infection, increased risk or incidence of fever, immunosuppression, reduced immune function, and reduced antibody production.

In some embodiments, a JAK1/2 inhibitor is administered to a subject receiving a therapy for a disease or disorder, and tumor number and/or tumor volume is reduced as compared to treatment with the therapy alone. In particular embodiments, a JAK1/2 inhibitor is administered to a subject receiving a therapy for a disease or disorder, and the number of cancer cells in the subject is reduced as compared to treatment with the therapy alone. In various embodiments, a JAK1/2 inhibitor is administered to a subject receiving a therapy for a disease or disorder, the probability of remission of the disease or disorder is decreased with the addition of the JAK1/2 inhibitor as compared to treatment with the therapy alone. In certain embodiments, a JAK1/2 inhibitor is administered to a subject receiving a therapy for a disease or disorder, and the probability of survival is increased as compared to the probability of survival from the therapy alone.

### EXAMPLES

### Example 1: PD-L1 and PD-L2 Gene Expression is Increased in Multiple-Myeloma Patients with Progressive Disease (PD)

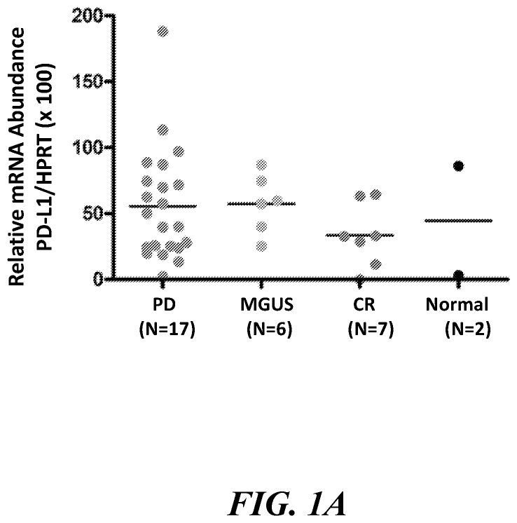

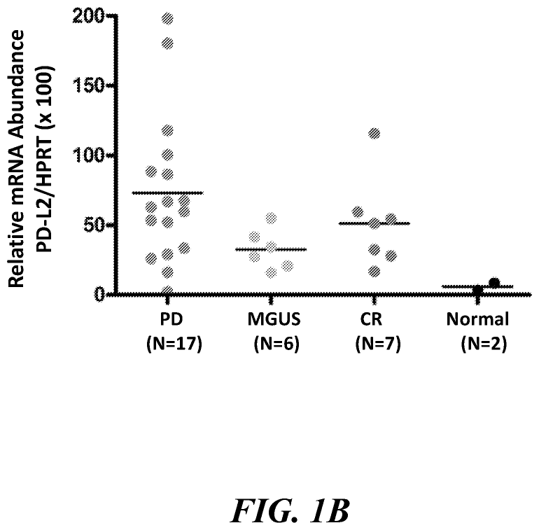

Bone marrow mononuclear cells (BMMCs) from multiple myeloma (MM) patients with progressive disease (PD) or in complete remission (CR) were isolated and analyzed using qPCR. We examined PD-L1 gene expression in MM patients with PD or in CR and those with monoclonal gammopathy of undetermined significance (MGUS) or healthy subjects (Normal). The results showed that PD-L1 gene expression measured at the mRNA level using quantitative PCR (qPCR) was markedly increased in BMMCs from MM patients with PD compared with those patients in CR or with MGUS. (FIG. 1A). PD-L2 gene expression was also increased among MM patients with PD compared with those patients in CR (FIG. 1B).

### Example 2: PD-L1 Gene Expression is Higher in CD138+ Myeloma Tumor Cells than CD138-Bone Marrow Mononuclear Cells (BMMCs)

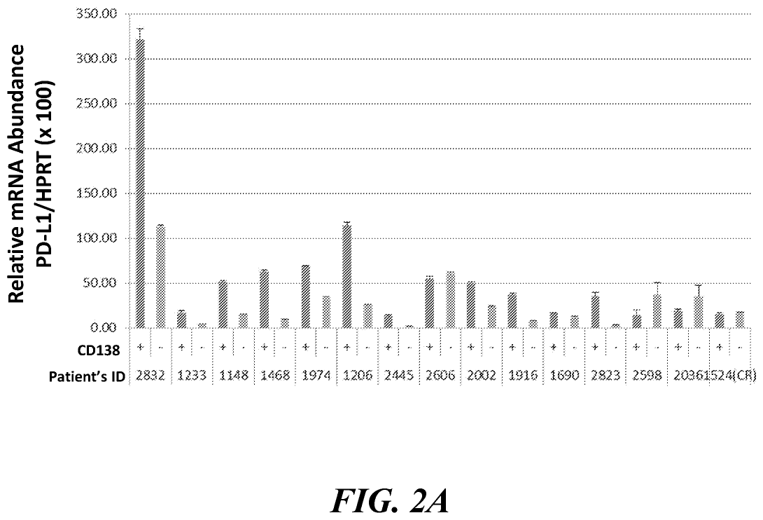

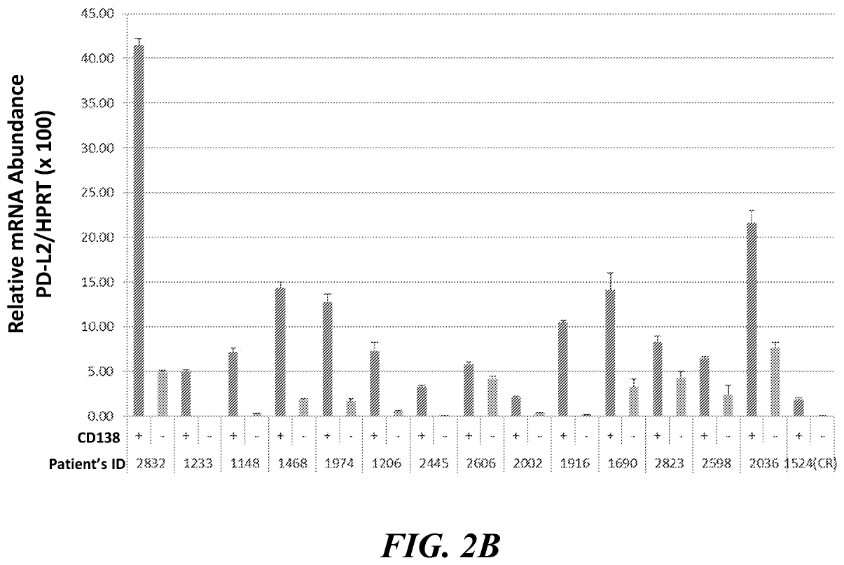

PD-L1 and PD-L2 gene expression was measured in CD138+ myeloma tumor cells and in CD138− mononuclear cells from the bone marrow of MM patients with PD (n=14) and in CR (n=1). The observed upregulation of PD-L1 in co-cultures with stromal cells indicated that upregulation occurs in nontumor cells within the patient-derived sample. CD138+ plasma cells were isolated by anti-CD138 antibody with magnetic beads and CD138− cells from bone marrow mononuclear cells were also collected for evaluation using a standard qPCR assay. PD-L1 (FIG. 2A) and PD-L2 (FIG. 2B) gene expression in CD138+ myeloma tumor cells was much higher than among CD138− mononuclear cells in the BMMCs. In the figure, + indicated the CD138+ cells and − indicates the CD138− cells.

### Example 3: PD-L1 and PD-L2 Expression in a MM Patient was Reduced by Co-Administration of Ruxolitinib, Methylprednisolone, and Lenalidomide

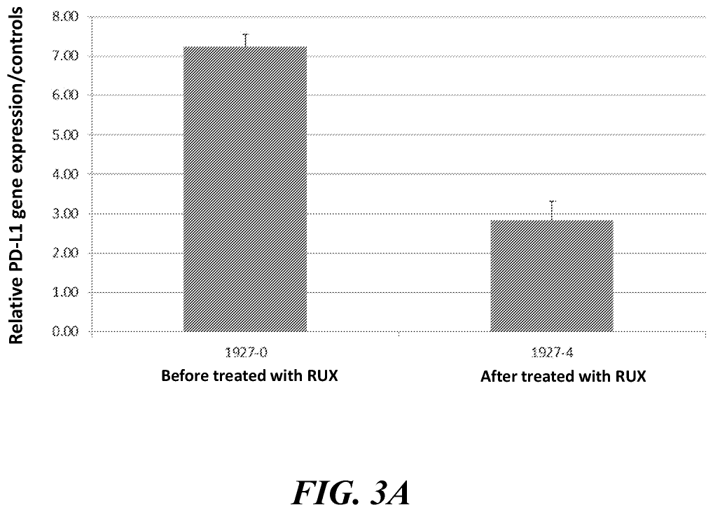

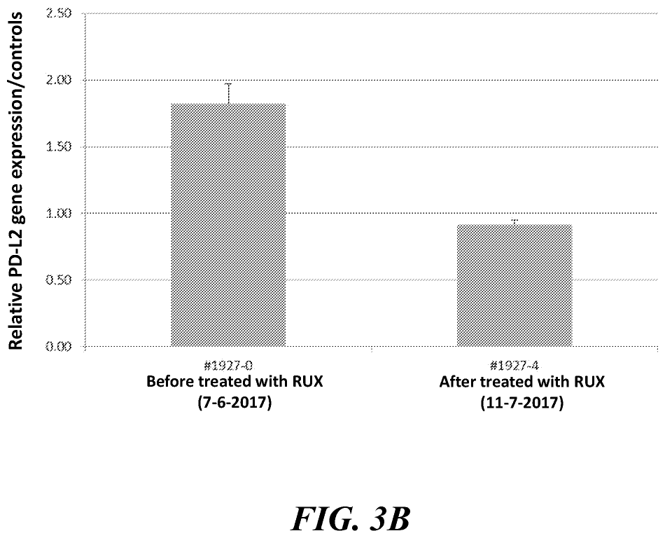

Both PD-L1 and PD-L2 gene expression was significantly reduced in bone marrow mononuclear cells (BMMCs) after 4 months of treatment with a RUX combination with other anti-myeloma agents (FIG. 3A and FIG. 3B). BMMCs were collected from a MM patient (who had previously treated and failed therapy with methylprednisolone and pomalidomide) before commencing treatment with ruxolitinib, methylprednisolone, and lenalidomide. A second sample of BMMCs was collected after treatment with ruxolitinib, methylprednisolone, and lenalidomide. PD-L1 (FIG. 3A) and PD-L2 (FIG. 3B) gene expression, measured by qPCR, were markedly reduced after the patient was treated with ruxolitinib, methylprednisolone and lenalidomide.

### Example 4: Ruxolitinib Reduces PD-L1 and PD-L2 Gene Expression in Bone Marrow Mononuclear Cells from MM Patient #3041 that are Co-Cultured with THP-1 Monocytes

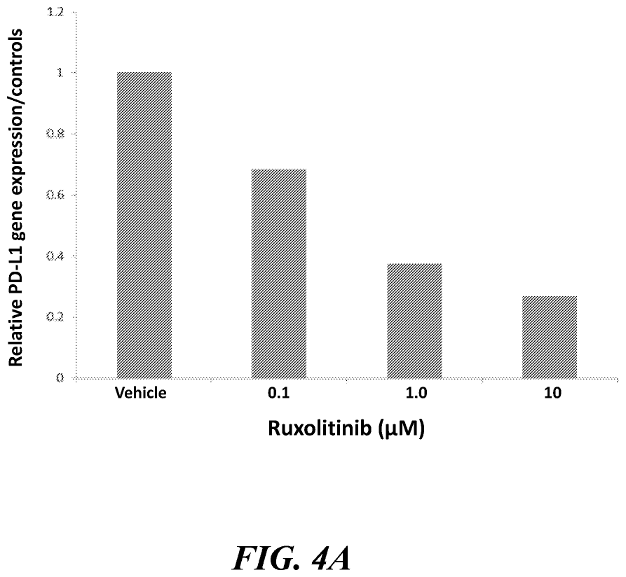

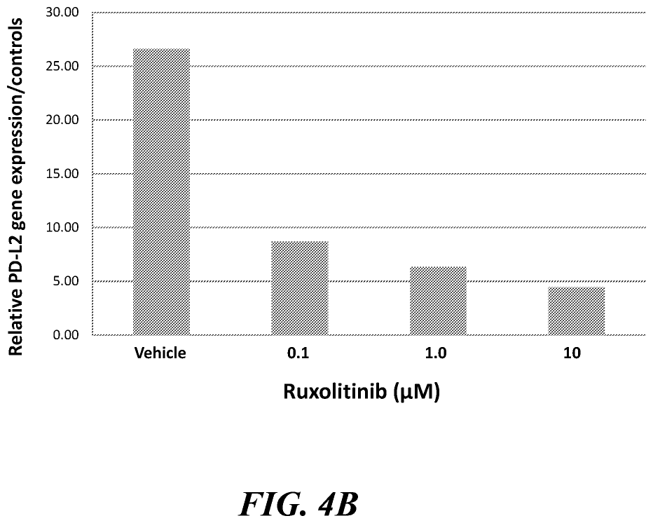

Next, we investigated the effects of ruxolitinib (RUX) on PD-L1 and PD-L2 gene expression in MM bone marrow mononuclear cells (BMMCs) co-cultured with THP-1 monocytes. THP-1 monocytes co-cultured with primary BMMCs from MM patient #3041 were grown in the presence or absence of RUX (1 μM). Using qPCR analysis, RUX treatment of MM BMMCs co-cultured with monocytes resulted in a marked decrease in both PD-L1 and PD-L2 gene expression in MM BMMCs (FIG. 4A and FIG. 4B).

### Example 5: Ruxolitinib Reduces PD-L1 Gene Expression in Bone Marrow Mononuclear Cells from MM Patient #2188 that are Co-Cultured with Stromal Cells or Monocytes

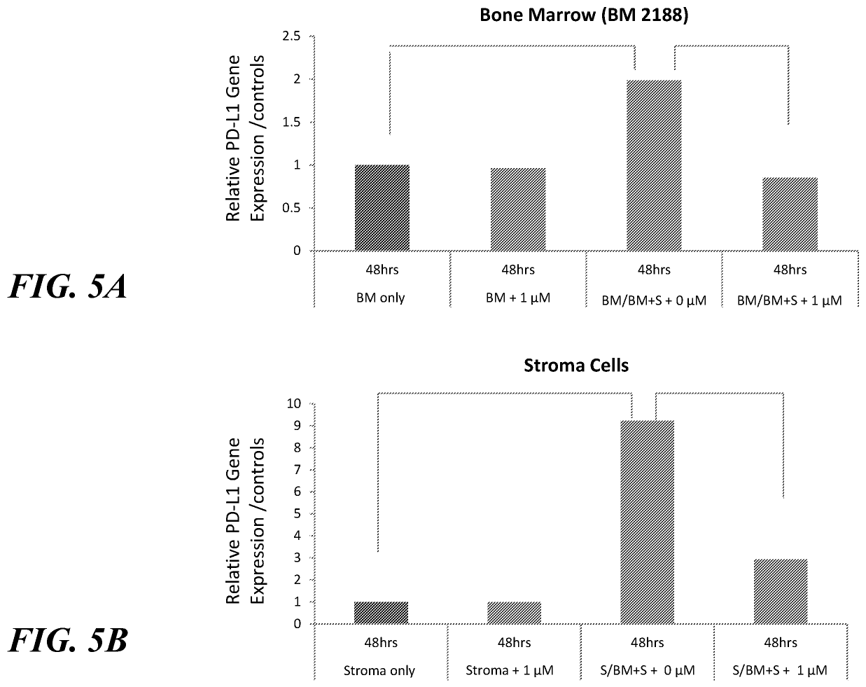

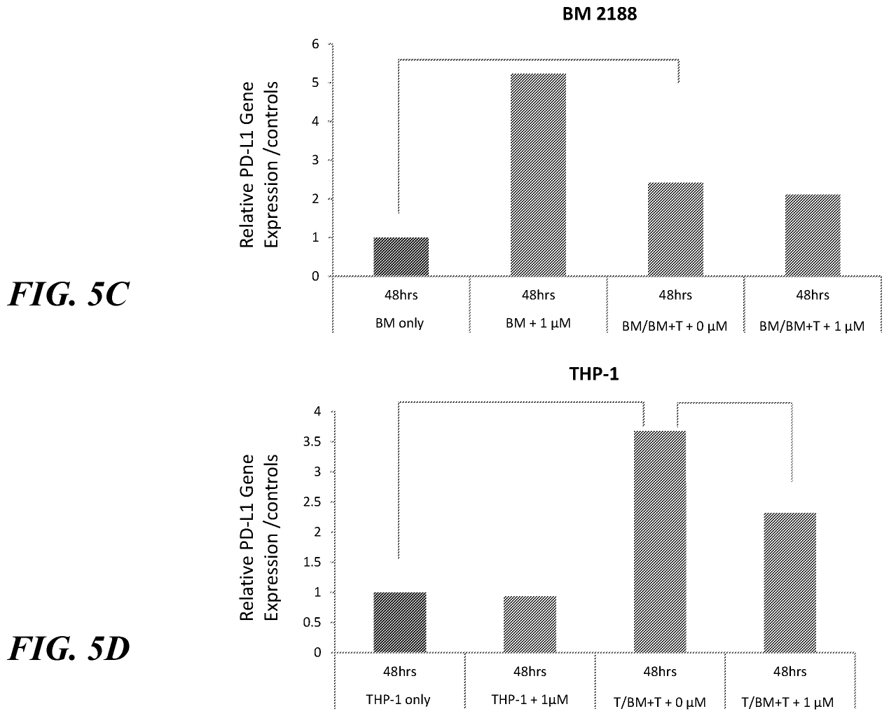

We also examined the effects of RUX on PD-L1 gene expression in MM tumor cells co-cultured with stromal cells or monocytes in vitro. BMMCs from MM patient #2188 were co-cultured with stromal cells from the HS-5 cell line (ATCC, CRL-11882) with or without RUX (1 μM) treatment. After 48 hours of co-culture, PD-L1 gene expression was increased in both BMMCs (FIG. 5A) and stromal cells (FIG. 5B) from the co-culture of both cell types compared with its expression in those cell types cultured alone. The increased PD-L1 levels were reduced in the presence of RUX (1 μM) in both cell populations after 48 hours of culture. Similarly, PD-L1 gene expression was increased in both BMMCs and monocytes (THP-1 cells) after co-culture compared with cells cultured alone (FIG. 5C and FIG. 5D) and reduced following exposure to RUX (1 μM).

FIG. 5A depicts relative PD-L1 gene expression in BMMCs from patient #2188 cultured with stromal cells (ATCC, HS-5) with RUX (1 μM) or without for 48 hours. BM, bone marrow cells alone; BM+1 μM, BMMCs treated with 1 μM RUX; BM/BM+S−0 μM, BMMCs with stromal cells without RUX; BM/BM+S+1 μM, BMMCs with stromal cells and 1 μM RUX.

FIG. 5B depicts relative PD-L1 gene expression in stromal cells (ATCC, HS-5) co-cultured with BMMCs from patient #2188 on Transwell inserts with (1 μM) or without RUX for 48 hours. Stroma cells (S) alone; stromal cells+1 μM, stromal cells treated with RUX (1 μM); S/BM+S−0 μM, BMMCs with stromal cells without RUX; S/BM+S−1 μM, BMMCs with stromal cells with RUX at 1 μM.

FIG. 5C depicts relative PD-L1 gene expression in BMMCs from patient #2188 co-cultured with THP-1 monocytes treated with (1 μM) or without RUX for 48 hours. BM, BMMCs alone; BM+1 μM, BMMCs treated with 1 μM RUX; BM/BM+T−0 μM, BMMCs with THP-1 monocytes without RUX; BM/BM+T+1 μM, BMMCs with THP-1 monocytes treated with RUX 1 μM.

FIG. 5D depicts relative PD-L1 gene expression in THP-1 monocytes co-cultured with BMMCs from patient #2188 on Transwell inserts with (1 μM) or without RUX for 48 hours. THP-1 only, THP-1 cells alone; THP-1+1 μM, THP-1 cells treated with 1 μM RUX; T/BM+T−0 μM, MM BMMCs with THP-1 cells without RUX; T/BM+T−1 μM, BMMCs with THP-1 monocytes with RUX (1 μM).

### Example 6: Ruxolitinib Decreases the Percentage of PD-L1-Expressing in Bone Marrow Mononuclear Cells and Increases the Percentage of Dead Cells

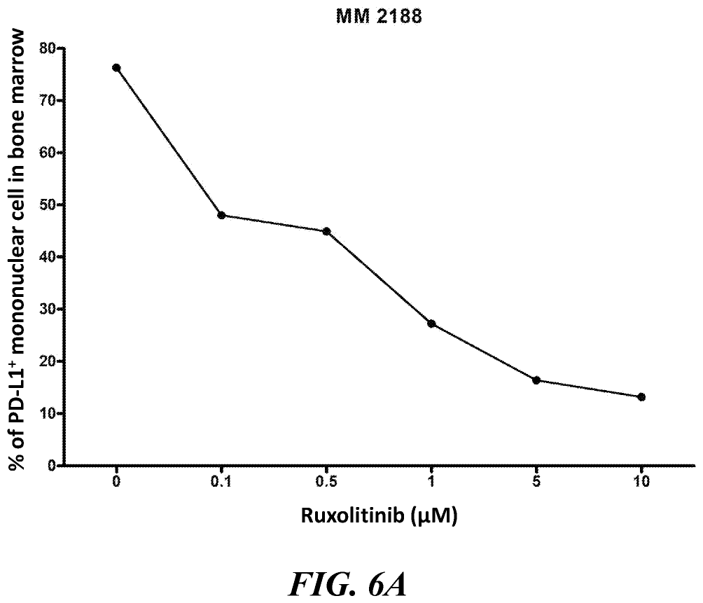

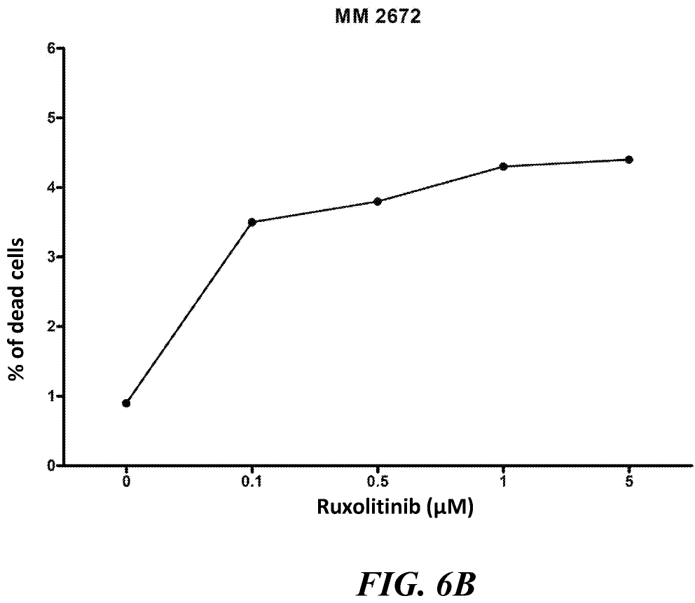

To investigate the effects of RUX on PD-L1 protein expression on myeloma cell membranes, fresh MM BMMCs were isolated from MM patients and co-cultured with stromal cells (HS-5, ATCC). After 72 hours, cells were fixed using 2% paraformaldehyde for 30 minutes on ice and washed with PBS twice. The cells were stained with anti-PD-L1 antibody conjugated with PE and anti-CD138 antibody conjugated with FITC antibody for 2 hrs and analyzed by flow cytometry using a Beckman Coulter FC500 cytometer with Cytomics CXP software (Beckman Coulter, Fullerton, Calif.). PD-L1-expressing MM tumor cells was reduced in a concentration dependent manner (FIG. 6A). The percentage of dead MM cells measured by Trypan blue staining was also increased in a concentration dependent fashion (FIG. 6B).

### Example 7: B7-H3 Gene Expression is Increased in MM Cells but Down-Regulated by Ruxolitinib Treatment

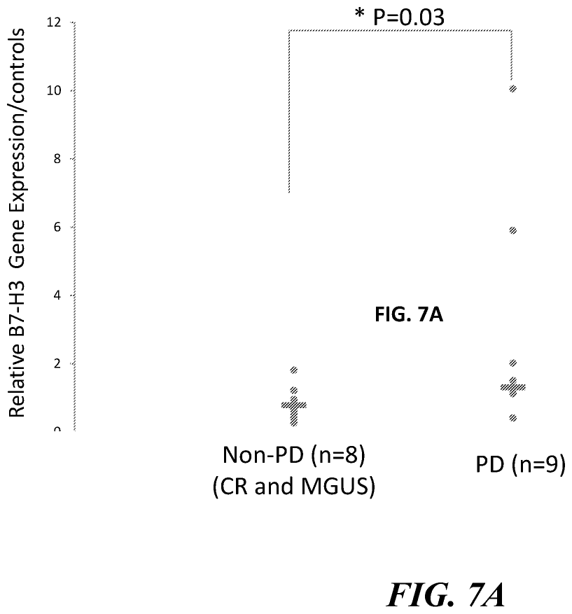

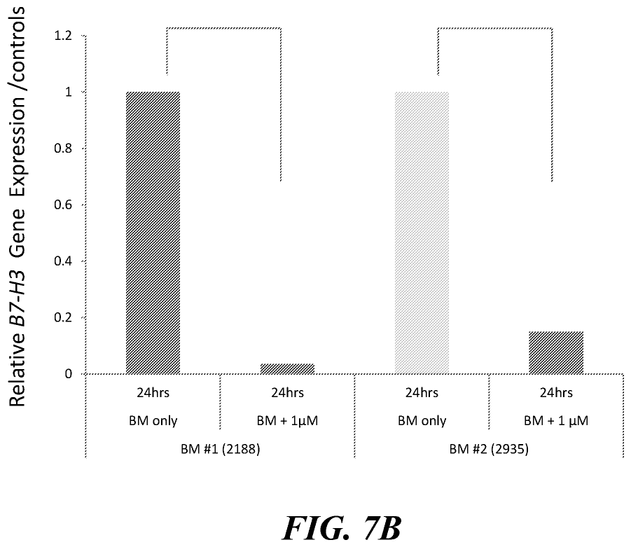

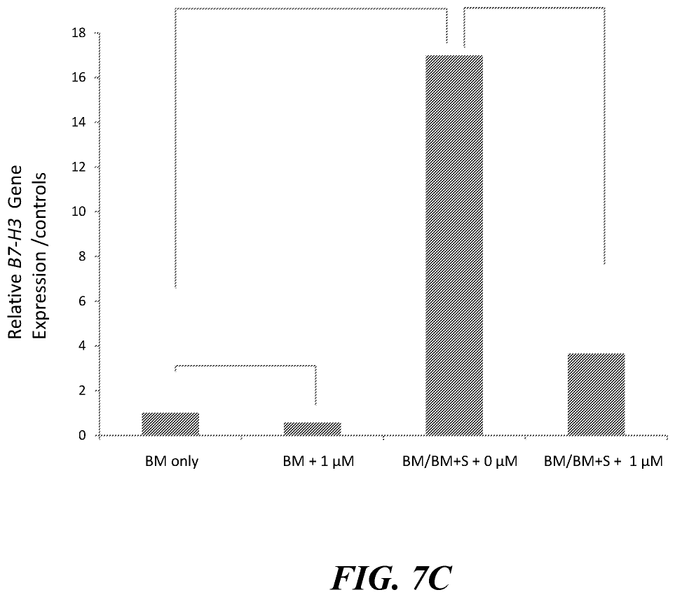

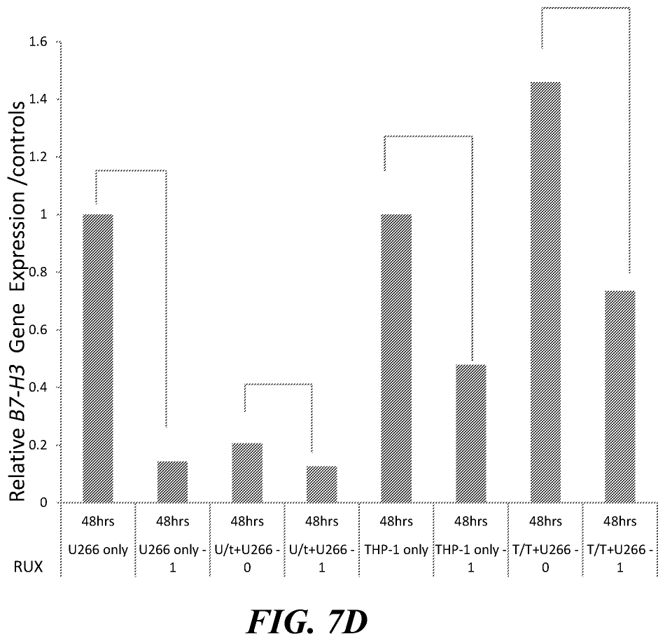

BMMCs from MM patients with PD or in CR or patients with MGUS were isolated and analyzed with qPCR. The results showed that B7-H3 gene expression was markedly increased in BMMCs from MM patients with PD compared with those patients in CR or MGUS. (FIG. 7A). B7-H3 gene expression was significantly increased in PD compared to non-PD patients (including CR and MGUS patients) using Mann-Whitney test (P<0.05). Each dot in FIG. 7A represents a subject, and the horizontal line represents the median of the group.

We further examined BMMCs from two MM patients (#s 2188 and 2935) treated with RUX (1 μM) for 24 hours. RUX reduced B7-H3 gene expression as measured using qPCR in BMMCs (FIG. 7B). We also analyzed BMMCs co-cultured with stromal cells. B7-H3 gene expression was increased 16-fold in BMMCs after these cells were co-cultured with stromal cells. RUX (1 μM) markedly reduced B7-H3 expression (FIG. 7C). We further determined the effect of RUX (1 μM) on gene expression of B7-H3 in the MM cell line U266 co-cultured with THP-1 monocytes (T) after 48 hours. RUX at 1 μM reduced B7-H3 expression in both U266 MM cells and THP-1 monocytes, cultured alone or co-cultured, compared to cell cultures not treated with RUX (FIG. 7D).

### Example 8: Ruxolitinib with Either an Anti-PD-L1 Antibody or Anti-PD-1 Antibody Increases T-Cell Induction of Apoptosis in Myeloma Tumor Cells

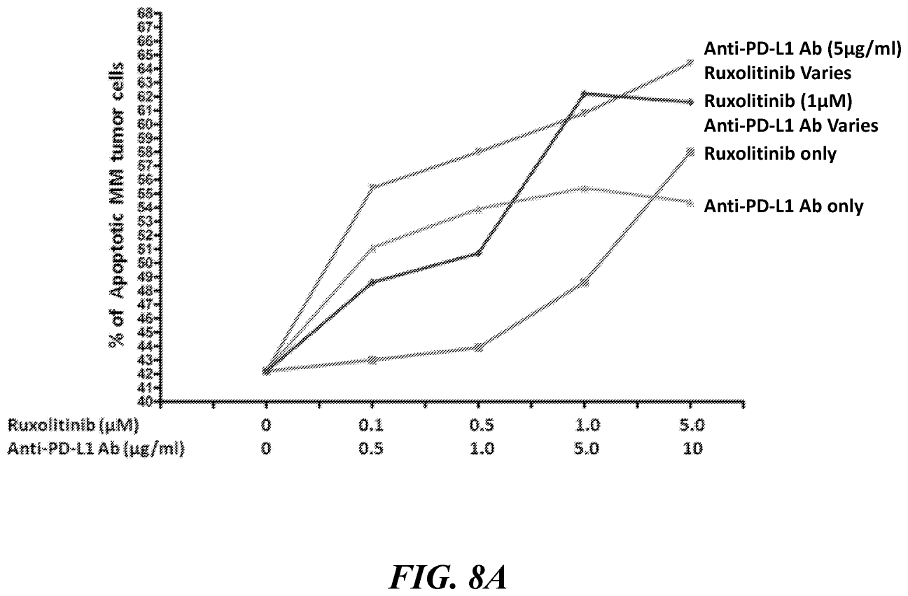

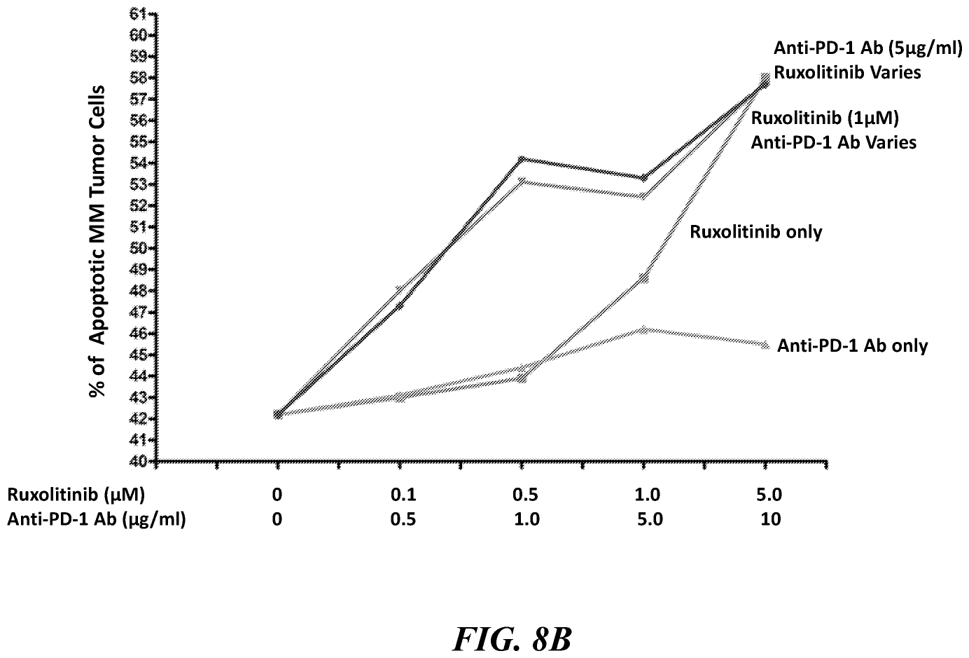

FIG. 8A demonstrates that the combination of ruxolitinib (RUX) and an anti-PD-L1 antibody (mouse monoclonal anti-PD-L1 antibody, Millipore Sigma MABC980) increases T-cell induction of apoptosis in myeloma tumor cells in vitro. T-cells (SUP-T1, ATCC) were pre-treated with IL-2 (20 ng/ml) for 24 hours. In the RUX at 1 μM with anti-PD-L1 group, the antibody concentrations were varied as indicated on the x-axis but the RUX was tested at a fixed concentration (1 μM). For the anti-PD-L1 at 5 mg/mL with RUX group, the RUX concentrations were varied as on the x-axis but the anti-PD-L1 antibody was evaluated at a fixed concentration (5 mg/mL). Tumor cells from the LAGκ-1A human MM xenograft were single-cell suspended and co-cultured with T cells treated with either RUX (1 μM) and anti-PD-L1 antibody or RUX alone for 72 hours. Tumor cell apoptosis was measured using the Annexin V assay per the manufacturer's protocol (Biovision) followed with flow cytometric analysis (FC-500 cytometer using Cytometric CXP software, both Beckman Coulter).

FIG. 8B demonstrates that the combination of ruxolitinib (RUX) and anti-PD-1 antibody (goat polyclonal anti-PD-1 antibody, R&D Systems AF1086) increases T-cell induction of apoptosis in myeloma tumor cells in vitro. T-cells were pre-treated with IL-2 (20 ng/ml) for 24 hours. For the RUX at 1 μM with anti-PD-1 group, the antibody concentrations were varied as indicated on the x-axis but the RUX was tested at a fixed concentration (1 μM). For the anti-PD-1 at 5 mg/mL with RUX group, the RUX concentrations were varied as on the x-axis but the anti-PD-1 antibody was evaluated at a fixed concentration (5 mg/mL). Myeloma tumor cells from the LAGκ-1A human xenograft were single cell suspended and co-cultured with T-cells treated with either RUX and anti-PD-1 antibody or RUX alone for 72 hours. Tumor cell apoptosis was measured using the Annexin V assay per the manufacturer's protocol (Biovision) followed with flow cytometric analysis (FC-500 cytometer using Cytometric CXP software, both Beckman Coulter).

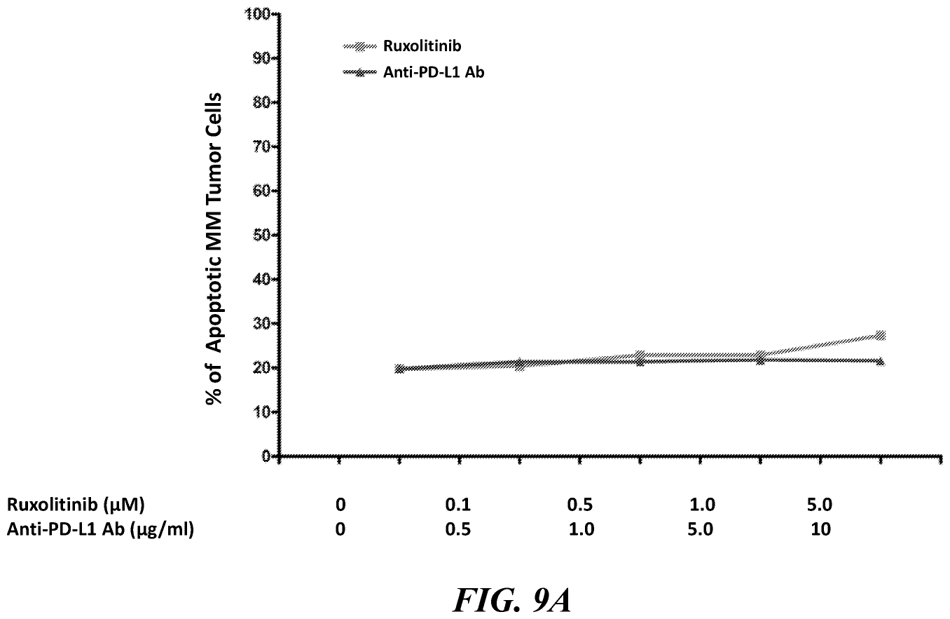

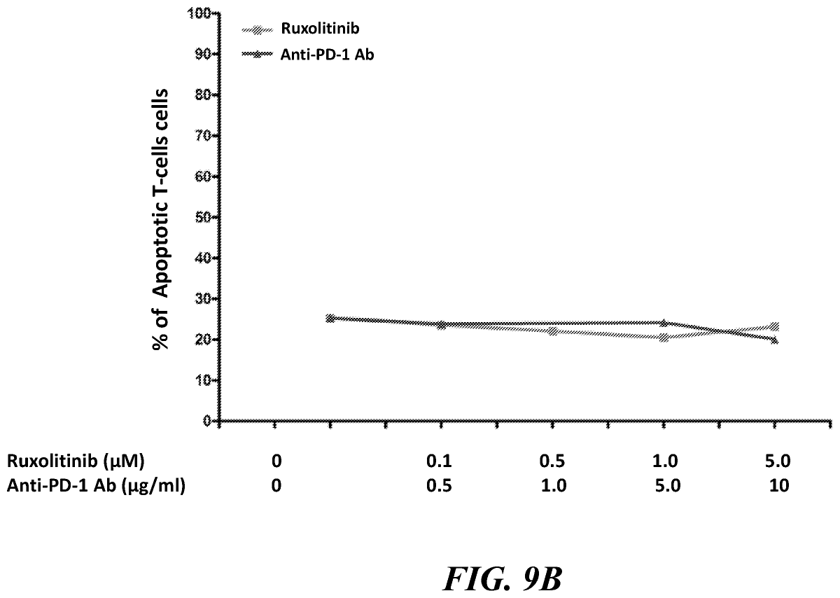

Control experiments with ruxolitinib (RUX), anti-PD-L1 antibody or anti-PD-1 antibody individually demonstrate that single-agent treatment does not lead to significant apoptosis of LAGκ-1A cells in the absence of T-cells. FIG. 9A depicts an apoptosis assay of myeloma tumor cells alone treated with either RUX or anti-PD-L1 antibody alone in vitro. RUX or anti-PD-L1 alone shows no significant anti-tumor effects on myeloma cells in the absence of T-cells. FIG. 9B depicts an apoptosis assay of myeloma tumor cells treated with either RUX or anti-PD-L1 antibody alone in vitro. RUX or anti-PD-L1 alone shows no significant anti-tumor effects on myeloma cells in the absence of T-cells.

### Example 9: Ruxolitinib Added to Activated T-Cells Increases T-Cell Induction of Apoptosis in CD138+ Myeloma Tumor Cells

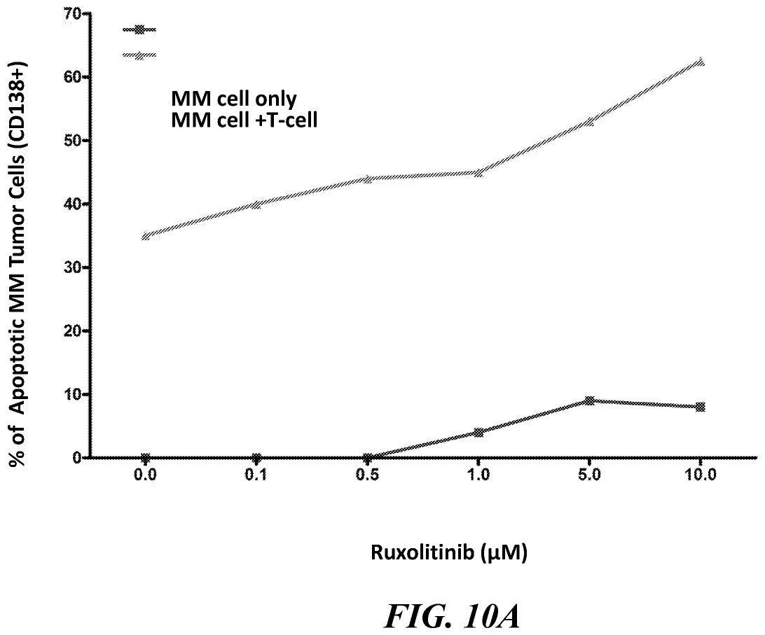

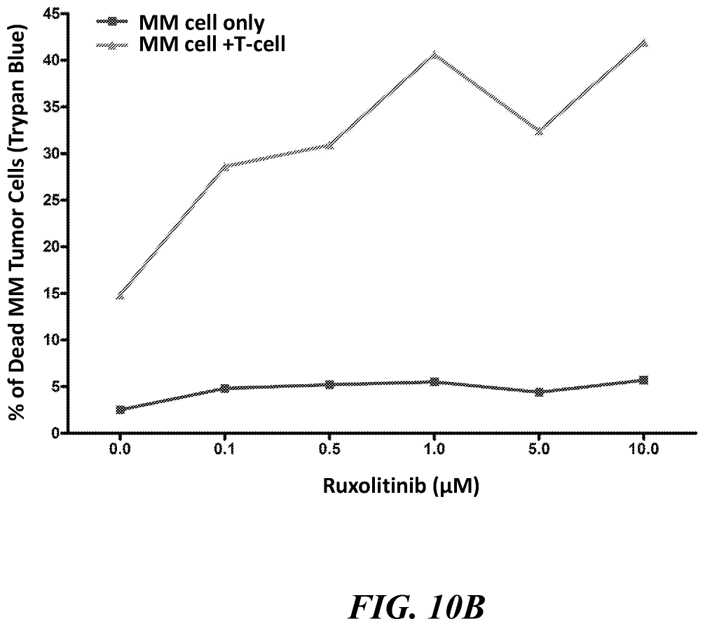

FIG. 10A depicts an apoptosis assay of fresh CD138-selected myeloma tumor cells combined with IL-2-stimulated T-cells (SUP-T1, ATCC) and treated with ruxolitinib (RUX) in vitro. T-cells were pre-treated with IL-2 (20 ng/ml) for 24 hours. Primary myeloma cells were CD138-selected using an immunoadsorption column and co-cultured with T cells with or without RUX at varying concentrations for 72 hours. Apoptosis in CD138+ cells was measured using the Annexin V assay per the manufacturer's protocol (Biovision) followed with flow cytometric analysis (FC-500 cytometer using Cytometrics CXP software, both Beckman Coulter). Minimal apoptosis was observed without T cells. Apoptosis occurred in a much higher proportion of myeloma tumor cells exposed to IL-2-stimulated T cells (FIG. 10A). RUX increased the fraction of cells that underwent apoptosis from approximately 35% up to about 65% in a concentration dependent fashion. Trypan blue staining demonstrated a similar effect of RUX on MM cell death as mediated by T cells (FIG. 10B). To summarize, Examples 8 and 9 have demonstrated that ruxolitinib enhances killing of MM cells by three immune-based therapies: anti-PD-L1 antibody, anti-PD-1 antibody and cytotoxic T cells.

### Example 10: Ruxolitinib Induces Increased IL-2 Expression in Myeloma Bone Marrow Mononuclear Cells

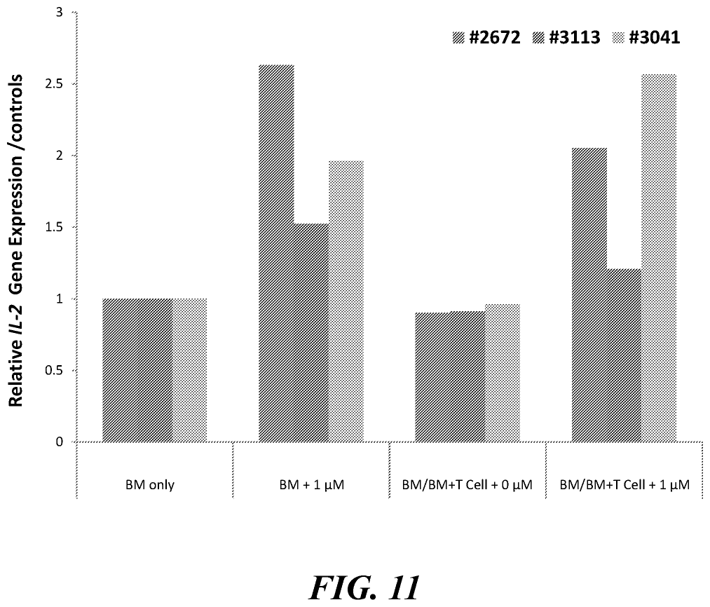

FIG. 11 demonstrates that RUX increased IL-2 gene expression in bone marrow mononuclear cells (BMMCs) from three MM patients. BMMCs from three MM patients were treated with RUX (1 μM) for 24 hours with or without co-culture with T cells in vitro. IL-2 gene expression levels were determined using qPCR RUX induced IL-2 expression in BMMCs alone and those co-cultured with T cells (SUP-T1, ATCC) in Trans-well culture dishes. These results show that RUX stimulates IL-2 expression in MM BMMCs, which should activate T cells and facilitate their anti-MM effects.

### Example 11: Ruxolitinib Reverses Checkpoint Inhibition by Downregulating PD-L1 and PD-L2 Expression on Both Tumor and Stromal Cells in Multiple Myeloma

Multiple myeloma (MM) tumor cells evade host immunity through the interaction of PD-L1 and PD L2 to PD-1 on T-cells. This creates an immunosuppressive milieu in the bone marrow (BM) microenvironment. The immune inhibitory proteins PD-L1 and PD-L2 are highly expressed in MM BM. Moreover, increased expression of these proteins are associated with resistance to treatment in MM. Ruxolitinib (RUX) is a JAK1/2 inhibitor that is effective for the treatment of myeloproliferative diseases. In this study, we examined PD-L1 and PD-L2 gene and protein expression in the BM of MM patients with progressive disease (PD) or in complete remission (CR). We further investigated the effects of RUX on expression of PD-L1 and PD-L2 in MMBM, and the effect of RUX in combination with anti-MM agents in vitro and in vivo.

BM mononuclear cells (MCs) and serum were collected from MM patients and healthy subjects after obtaining IRB approval. Single-cell suspensions were prepared from human MM LAGκ-1A xenografts which had been grown in the mice. The cells were cultured and treated with or without RUX and then were determined by qPCR, flow cytometric analysis, ELISA, and western blot.

The results from qPCR and flow cytometric assays showed that PD-L1 and PD-L2 gene expression was markedly increased in BMMCs from MM patients with PD compared with patients in CR or with healthy controls. We further investigated the effects of RUX on PD-L1 and PD-L2 expression in primary and stromal cells from MM patients' BM samples in vitro. RUX treatment markedly reduced PD-L1 and PD-L2 gene and protein expression in the MM tumor cells cultured alone or co-cultured with stromal cells in a concentration dependent pattern. We then determined whether RUX can augment the anti-MM effects of T-cells in vitro. RUX (0, 0.1, 0.5, 1, and 5 μM) increased MM cell apoptosis in the presence of IL-2 stimulated T-cells in a concentration dependent fashion, to a similar degree to anti-PD-1 (0, 0.5, 1, 5, and 10 μg/ml) or anti-PD-L1 (0, 0.5, 1, 5, and 10 μg/ml) antibody treatment. Moreover, the combination of RUX with anti-PD-1 or anti-PD-L1 antibody increased T-cell-induced MM cell apoptosis more than the agents alone. To evaluate the efficacy of drugs in vivo, severe combined immune deficient mice implanted with the human MM xenograft LAGκ-2 were treated with RUX (30 mg/kg). The results showed PD-L1 expression in the xenograft was significantly decreased in RUX-treated mice compared with the untreated control group. In contrast, RUX had no effect on PD-1 expression on T-cells.

The PD-L1/PD-1 pathway delivers inhibitory signals that regulate both peripheral and central tolerance and inhibit anti-tumor immune-mediated responses. This study demonstrated that the JAK inhibitor RUX downregulated PD-L1 and PD-L2 expression in both MM tumor and stromal cells. We also demonstrated that RUX alone increased T-cell-induced apoptosis of MM cells; and, moreover, the combination of RUX with anti-PD-1 and anti-PD-L1 further increased apoptosis. The results suggest that JAK inhibitors may be effective for treating MM patients through their ability to reduce expression of checkpoint proteins involved in the development of immune resistance. Thus, JAK inhibitors should help overcome the immune resistance generated by these proteins for patients with this B-cell malignancy.

### Example 12: The JAK1/2 Inhibitor Ruxolitinib Downregulates the Immune Checkpoint Protein B7-H3 in Multiple Myeloma

The JAKS TAT pathway plays a critical role in the regulation of hematopoietic pathways and immunological cytokine signaling. The JAK pathway is also involved in tumor cell proliferation and drug resistance in multiple myeloma (MM). Thus, inhibition of the JAK pathway should be a potentially effective strategy for treating MM patients. B7-H3 is an immune checkpoint protein in the B7 superfamily and has been shown overexpressed in several tumors. Immune checkpoint blockade may suppress tumor progression or enhance anti-tumor immune responses. In this study, we investigated the effects of the JAK1/2 inhibitor ruxolitinib (Rux) on B7-H3 in MM.

Bone marrow mononuclear cells (BMMCs) were collected from MM patients after obtaining IRB approval. Single-cell suspensions were prepared from human MM LAGλ-1A xenografts which had been grown in severe combined immunodeficient mice. HS-5 stromal and SUP-T1 T cells were purchased from ATCC. The cells were cultured and treated with or without RUX and then subjected to qRT-PCR, flow cytometric analysis, and western blot analysis. For qRT-PCR, total RNA was extracted and applied to cDNA synthesis, followed by qPCR. Gene expression was analyzed in MM BMMCs alone or co-cultured with stromal cells or T cells with or without Rux treatment (1 μM) in vitro.

We identified increased B7-H3 expression in MMBMMCs from patients with progressive disease (PD) patients compared to those in complete remission (CR). Rux significantly reduced B7-H3 expression in MMBMMCs in patients with PD, MM cells (U266), and BM from patients in PD when co-cultured with stromal cells (HS-5) after 48-72 hours. Rux decreased B7H3 expression in the human MM xenograft model LAGλ-1A when cultured ex vivo. In addition, Rux suppressed B7-H3 at protein levels as shown with flow cytometric analysis and western blotting, consistent with the gene expression results.

Next, we tested whether B7-H3 blockade by Rux could potentially restore exhausted T cell activity against myeloma cells in MMBM. We found that Rux can increase IL-2 and CD8 gene expression in MMBM with lower plasma percentages (<30%) but not among those with higher plasma cell percentages (>70%). Rux also elevated IL-2 and CD8 gene expression in BM when it was cocultured with T cells (SUP-T1), suggesting Rux may mediate immunological cytokine signaling. B7-H3-neutralizing antibody increased CD8 gene expression in MMBM in vitro, suggesting that one of the mechanisms through which Rux upregulates CD8 T cells in MMBM may be via downregulation of B7-H3.

The immune checkpoint protein B7-H3 is overexpressed in MMBM in PD compared to CR patients. The JAK1/2 inhibitor Rux can decrease B7-H3 expression and increase IL-2 and CD8 expression in BM in vitro. Our results provide evidence for Rux inhibiting the immune checkpoint protein B7-H3 which may potentially restore exhausted T-cell activity in the MMBM tumoral microenvironment.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention contemplated herein.

In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestions that they constitute valid prior art of form part of the common general knowledge in any country in the world.

What is claimed is:

1. A method of inhibiting cancer cell growth, comprising contacting the cancer cell with a JAK1/2 inhibitor or derivative thereof and an immune-based therapy.

2. A method of decreasing expression of a checkpoint receptor or ligand by a cell, comprising contacting the cell with a JAK1/2 inhibitor or derivative thereof and an immune-based therapy.

3. A method of treating and/or inhibiting cancer in a subject being treated for a cancer with an immune-based therapy, comprising administering the subject an immune-based therapy and a JAK1/2 inhibitor or derivative thereof.

4. A method of increasing the efficacy of an immune-based therapy in a subject being treated for a cancer, comprising administering the subject a JAK1/2 inhibitor or derivative thereof in addition to the immune-based therapy being provided to the subject.

5. The method of any one of claims 1 to 4, wherein the JAK1/2 inhibitor is selected from the group consisting of ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, and CHZ868.

6. The method of claim 5, wherein the JAK1/2 inhibitor is ruxolitinib.

7. The method of any one of claims 1 to 6, wherein the immune-based therapy is a cell-based therapy.

8. The method of claim 7, wherein the cell-based therapy is selected from the group consisting of a group consisting of CAR T-cell therapy, T-cell therapy, donor lymphocyte infusion, allogeneic hematopoietic cell therapy, autologous hematopoietic cell therapy, and natural killer (NK) cell therapy.

9. The method of any one of claims 1 to 6, wherein the immune-based therapy is selected from the group consisting of a bispecific T-cell engager (Bi1E) therapy, a monoclonal antibody-based therapy, an antibody-drug conjugate, a PD-1 inhibitor, a PDL-1 inhibitor, a PD-L2 inhibitor, a B7-H3 inhibitor, a CTLA-4 inhibitor, an immunoreceptor tyrosine-based inhibition motif (ITIM) inhibitor, and an immunoreceptor tyrosine-based activation motif (ITAM) stimulatory agent.

10. The method of any one of claims 1 to 6, wherein the immune-based therapy is selected from the group consisting of pembrolizumab, nivolumab, cemiplimab, atezolizumab, avelumab, and durvalumab.

11. The method of any of claims 1 to 10, wherein the cancer is a hematological malignancy or the cancer cell is derived from a hematological malignancy.

12. The method of claim 11, wherein the hematological malignancy is a B-cell condition or disorder selected from the group consisting of: multiple myeloma (MM), Waldenstrom's macroglobulinemia (WM), chronic lymphocytic leukemia (CLL), B cell non-Hodgkin's lymphoma, plasmacytoma, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B cell lymphoma, splenic lymphoma, mantle cell lymphoma, large cell lymphoma, diffuse mixed cell lymphoma, immunoblastic lymphoma, primary mediastinal B cell lymphoma, pulmonary B cell angiocentric lymphoma, small lymphocytic lymphoma, B cell proliferations of uncertain malignant potential, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

13. The method of claim 11, wherein the cancer is multiple myeloma or the cancer cell is a multiple myeloma cell.

14. The method of claim 13, wherein the multiple myeloma is relapsed or refractory multiple myeloma.

15. The method of any one of claims 1 to 14, wherein the cancer is characterized by upregulation of PD-1, PD-L1, PD-L2, and/or B7-H3.

16. The method of any one of claims 3 to 15, wherein the JAK1/2 inhibitor is intravenously administered to the subject.

17. The method of any one of claims 3 to 15, wherein the JAK1/2 inhibitor is orally administered to the subject.

18. The method of any one of claims 3 to 17, wherein the subject is being treated with, or has been previously treated with radiation therapy, chemotherapy, transplantation, immunotherapy, hormone therapy, or photodynamic therapy.

19. A pharmaceutical composition comprising a JAK1/2 inhibitor and an immune-based therapy.

20. A kit comprising a JAK1/2 inhibitor, an immune-based therapy, and instructions for use thereof.

21. A JAK1/2 inhibitor for use in the treatment of a cancer characterized by upregulation of one or more of the checkpoint proteins PD-1, PD-L1, PD-L2, and B7-H3.

22. The method of any one of claims 1 to 18, the pharmaceutical composition of claim 19, the kit of claim 20, or the JAK1/2 inhibitor of claim 21, provided that the immune-based therapy is not nivolumab or pembrolizumab.
